(19) United States
(12) Patent Application Publication
(10) Pub. No.: US 2010/0227827 A1
(43) Pub. Date: Sep. 9, 2010

(54) METHOD OF INCREASING TRYPTOPHAN AND NICOTINAMIDE LEVELS IN VIVO

(76) Inventor: Ronald W. Pero, Lund (SE)

Correspondence Address: FOX ROTHSCHILD LLP;PRINCETON PIKE CORPORATE CENTER, 997 LENOX DRIVE, BLDG. #3, LAWRENCEVILLE, NJ, 08648, US

(21) Appl. No.: 12/373,478

(22) PCT Filed: Jul. 11, 2007
(86) PCT No.: PCT/US07/73261

§ 371 Date: Jan. 14, 2010

Related U.S. Application Data
(60) Provisional application No. 60/819,624, filed on Jul. 11, 2006.

(51) Int. Cl. A61K 31/19; C07C 62/00; A61K 31/44; A61K 31/192; A61K 31/341; A61K 31/59; A61K 31/355; A61K 31/7048; A61K 31/352; G01N 33/68; C12Q 1/02; A61P 25/24; A61P 29/00; A61P 37/04; A61P 35/00; A61P 3/04

(52) U.S. Cl. 514/27; 562/508; 514/557; 514/355; 514/570; 514/474; 514/167; 514/458; 514/456; 436/89; 435/29

(57) ABSTRACT

Novel methods of increasing the biosynthesis of aromatic amino acids and compositions containing the precursors of said aromatic amino acids or nicotinamide and methods of use thereof. In addition, methods of monitoring the therapeutic effects of an anti-oxidant therapy by measuring serum protein thiols as a surrogate or predictor of such therapy are disclosed.

### CROSS-REFERENCE TO RELATED APPLICATIONS

This application claims the benefit of the U.S. Provisional Application No. 60/819,524 filed on Jul. 11, 2006. This application is also related to the international application PCT/US2006/009394 filed on Mar. 16, 2006, which is herein incorporated in its entirety.

### FIELD OF THE INVENTION

The present invention relates to the methods of increasing the biosynthesis of aromatic amino acids and compositions containing the precursors of said aromatic amino acids or nicotinamide and methods of use thereof. In addition, methods of monitoring the therapeutic effects of an anti-oxidant therapy by measuring serum protein thiols as a surrogate or predictor of such therapy are disclosed.

### BACKGROUND OF THE INVENTION

The therapeutic effects of Uncaria tomentosa (hereinafter “Cat's Claw”) is well known. The Ashanika Indians have used the Cat's Claw extracts in water to help control infections, inflammatory disorders and even mental states. Cat's Claw can also be combined with other ingredients such as huchuhuasi bark, capsaicin, burdock root, sheep sorrel or slippery elm bark to improve its therapeutic efficacy. The active ingredients in Cat's Claw include proanthocyanidins, quinovic acids and glycosides thereof, oxindole alkaloids (pteridine, isopteridine, uncarine, mitraphylline, isomitraphylline), N-oxide, rhynocophylline, carboline alkaloid, hirustine, N-oxide triterpenes, polyphenols, phytosterosols (stigmasterol and campesterol). (Senatore, A., et al., Phytochemical and Biological Study of Uncaria tomentos, Boll Soc Ital Biol Sper. 1989; 65(6):517-20)).

The present inventor, professor Ronald W. Pero was the first to identify quinic acid analogs as the primary bioactive component of hot water extracts of Cat's Claw. (Sheng, Y., Akesson, C., Holmgren, K., Bryngelsson, C., Giampapa, V., Pero, R. W., An active ingredient of Cat's Claw water extracts. Identification and efficacy of quinic acid. Journal of Ethanopharmacology 96(3): 577-584, 2005). U.S. Pat. Nos. 6,039,949, 6,238,675, and 6,361,805, hereby incorporated by reference in their entirety, describe water soluble extracts of the plant species Cat's Claw having a high degree of the anti-tumor, inflammatory and immune stimulatory activities. Preparation of Cat's Claw extracts described in said patent have been associated with various therapeutic uses and is available commercially under as C-Med-100®. C-Med-100® is a 100% bioavailable hot water extract of Cat's Claw. Similar formulations are also available under different trade names such as AC-11™ and Protectagen™.

United States published patent application no. 20050176825 discloses methods for the isolation, purification, and identification of quinic acid and quinic acid salts as the active ingredients of C-Med-100® in vivo. This patent application also discloses the use of quinic acid and its salts and chelates to treat disorders associated with the immune system, inhibit inflammatory response, treat disorders associated with inflammatory response, enhance the DNA repair process, enhance the anti-tumor response, and treat disorders associated with the response to tumor formation and growth.

Other known medical uses of quinic acid and its analogs are U.S. Pat. No. 5,656,665 and U.S. Pat. No. 5,589,505 which disclose the treatment of skin wrinkles and U.S. Pat. No. 6,111,132 and U.S. Pat. No. 6,225,341 which disclose the treatment of flu as a neuroamidase inhibitor.

Microflora of the GI tract have a functional shikimate pathway that can metabolize quinic acid to hippuric acid via benzoic acid (Adamson, R. H., Bridges, J. W., Evans, M. E., Williams, R. T., Species differences in aromatization of quinic acid in vivo and the role of gut bacteria, Biochem Jour 116:437-433, 1970). Consumption of both black tea and green tea results in an increase in the excretion of hippuric acid into urine. The only known previously documented indicator of quinic acid metabolism (i.e. hippuric acid) varied greatly from animal to animal rendering this metabolite an unreliable quantitative estimate of quinic acid exposure. Although quinic acid can be metabolized to hippuric acid in the gut, the primary metabolic source of hippuric acid is thought to be the liver (Krähenbühl, L., Reichen, J., Talos, C., Krähenbühl, S., Benzoic acid metabolism reflects hepatic mitochondrial function in rats with long-term extrahepatic cholestasis, Hepatology 25 (2): 259-508, 1997).

Given the above, it is apparent that higher animals are generally incapable of synthesizing aromatic amino acids, and must rely on their diet to meet their needs for such amino acids. Accordingly, there is a need in the art for new therapeutic approaches that can optimize the desired serum concentrations of aromatic amino acids in higher animals.

### SUMMARY OF THE INVENTION

The inventor have surprisingly found that since there was no quinic acid elevation in peripheral circulation of humans following the oral administration of the instantly claimed compounds, the compounds of the present invention are effective synthetic precursors for the production of tryptophan and nicotinamide in the gastro-intestinal (GI) tract via the shikimate pathway. Raised tryptophan and nicotinamide levels mediate a broad spectrum of health benefits such as anti-oxidant, serotonin-mediated, dopamine-mediated, and NAD mediated activities.

An aspect of the present invention is directed towards a precursor for an aromatic amino acid having the following formula:
A-(X)ₙ where A is selected from the group consisting of

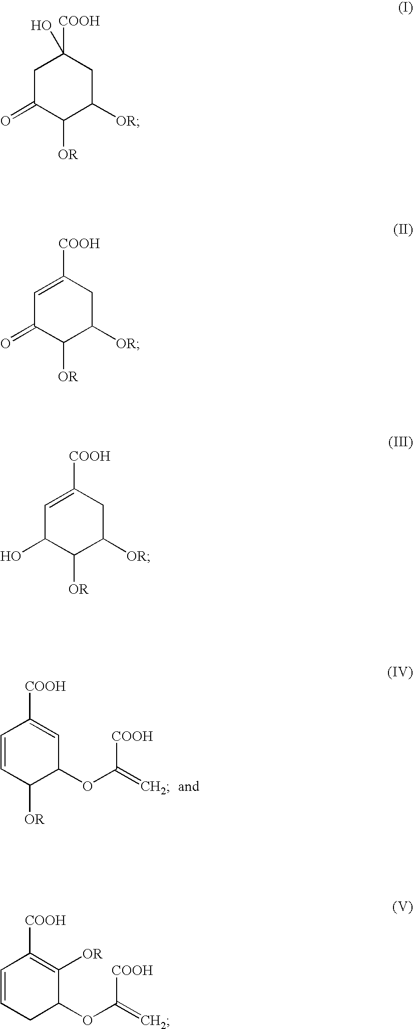

and anions, esters thereof; wherein R is H or C₁-C₃ alkyl, and
X is selected from the group consisting of any monovalent, divalent, trivalent anions that can form salts or hydroxides or amines, and
“n” is an integer or a fraction, wherein 1<n=10.

In a preferred embodiment the aromatic amino acid is selected from the group consisting of tryptophan, phenylalanine, tyrosine and phenylephederine.

Another aspect of the present invention is directed towards a composition comprising (a) therapeutically effective amounts of a compound selected from the group consisting of above compounds (I); (II); (III); (IV) and (V);
wherein R is H or C₁-C₃ alkyl; and anions and esters thereof, and

A-(X)ₙ (VI)
wherein A, X and n are as defined above; and
(b) a pharmaceutically acceptable vehicle.

In a further aspect the composition comprises therapeutically effective amounts of primary antioxidants. In an embodiment the primary anti-oxidant is a flavonoid.

Another aspect of the invention is directed towards an animal model methodology for identifying an effective anti-oxidant therapeutic regimen for a disease state characterized by aromatic amino acid deficiency comprising the steps
(a) obtaining a mammal selected from the group consisting of mice, rats, rabbits, dogs, pigs, monkeys and humans
(b) determining a baseline value ratio for urinary tryptophan, nicotinamide, hippuric, kynurenine levels/tissue or blood thiol(s) levels of said mammal,
(c) administering to the mammal a composition comprising a precursor of an aromatic amino acid,
(d) determining a value for the ratio of urinary tryptophan, nicotinamide, hippuric, kynurenine levels/tissue or blood thiol(s) levels of said mammal after a cycle of the anti-oxidant treatment,
(e) comparing the values obtained in steps (b) and (d),
wherein an increase over the baseline value indicates that the therapeutic regimen has been effective.

Another aspect of the present invention is directed towards a method of testing the in vivo oxidative stress in a patient in need comprising
(a) treating the patient with a composition comprising a precursor for an aromatic amino acid,
(b) obtaining a sample of protein thiols from said patient,
(c) determining the protein thiol(s) levels in the sample,
(d) measuring urinary levels of tryptophan or nicotinamide,
(e) analyzing the thiol(s) levels in sample of step (c) in relation to the urinary levels of tryptophan or nicotinamide obtained in step (d) expressed as (d)/(c),
wherein an increase in the value of said ratio is a predictor of low oxidative stress and down regulation of DNA repair.

Another aspect of the present invention is directed towards a method of ascertaining a patient's susceptibility to a disease characterized by increased level of oxidative stress comprising
(a) treating the patient with a composition comprising an aromatic amino acid precursor,
(b) obtaining a body sample from said patient comprising protein thiol(s) level,
(c) determining the protein thiol(s) level of the sample,
(d) measuring urinary levels of tryptophan or nicotinamide levels,
(e) analyzing the thiol(s) levels in sample of step (c) against the urinary levels of tryptophan or nicotinamide obtained in step (d) wherein a higher value against a predetermined normalized value (d)/(c) obtained from a population suffering from said disease is the predictor of said individual to oxidative stress and down regulation of DNA repair.

Another aspect of the present invention is directed towards a method of treating a condition characterized by deficiency in serum aromatic amino acid levels comprising administering an aromatic amino acid precursor and a pharmaceutically acceptable vehicle. In this aspect of the invention, the compositions employed are preferably for oral administration and are combinable with various types of food, snacks, meal products as well as beverages.

Another aspect of the present invention is directed towards methods for determining the risk of developing a disease characterized by deficiency in aromatic amino acids in an individual in need thereof, comprising the steps of:
(a) obtaining levels of the individual's serum thiol(s) and levels of urinary tryptophan, nicotinamide or combinations or both,
(b) establishing a value for the ratio of urinary tryptophan, nicotinamide or combination thereof/serum thiols for said individual,
(c) comparing the value of step (b) against a predetermined normalized levels of the same obtained from a population afflicted by said disease, wherein
said comparison indicates the baseline value for the oxidative stress in said individual.

This methodology can further comprise steps (d) administering to the individual during a treatment cycle a composition comprising a precursor for an aromatic amino acid, and/or an antioxidant (e) obtaining levels of the individual's serum protein thiol(s) and levels of urinary tryptophan, nicotinamide, hippuric acid, or all, after the end of the treatment cycle, and (f) establishing a value for the ratio of urinary tryptophan, nicotinamide or combination thereof/serum protein thiol(s) for the individual, after said treatment cycle.

### BRIEF DESCRIPTION OF THE DRAWINGS

FIG. 1. Illustrates the pharmacokinetics of a single high dose of quinic acid at 6000 mg administered orally in distilled water, and then within 15 min 30 ml serum samples collected at the indicated times. Determination of serum quinic acid in relation to its main metabolite hippuric acid, were performed using High Pressure Liquid Chromatography (HPLC) as described in Material and Procedures section. To visualize the maximum time of uptake, the data were presented as a percentage of maximum uptake: quinic acid=100% at 12.5 hr (4400 μg/ml); hippuric acid=100% at 7 hr (55 μg/ml).

FIG. 2. Illustrates the urinary profile of subject RP treated with 3000 mg/day Aqua Bimini™ (quinic acid ammonium chelate) for 36 days and then followed for an additional 8 months with no further treatment. Urinary hippuric and quinic acid levels (mM) were estimated on the sampling dates indicated.

FIG. 3. Illustrates the urinary profile of subject HL treated with 1500 mg/day Aqua Bimini™ (quinic acid ammonium chelate) from May 1-31, 2006 and then followed for an additional 8 months with no further treatment. Urinary hippuric and quinic acid levels (mM) were estimated on the sampling dates indicated.

FIG. 4. Illustrates serum levels of hippuric acid in subject HL and subject RP treated daily with 1500 or 3000 mg/day Aqua Bimini™ (quinic acid ammonium chelate) for 36 days, and then followed-up (no treatment) from 30 days more. Total sampling points during this period were: subject RP: n=12, subject HL: n=12, p<0.01.

FIG. 5. Illustrates the dependence of urinary levels of hippuric acid, the primary known metabolite of quinic acid, on the urinary level of quinic acid in untreated individuals and in subjects treated with 1500 mg/day and 3000 mg/day Aqua Bimini™ (quinic acid ammonium chelate) for 30 day, then followed for an additional 8 month immediately after treatment. These data were statistically analyzed as a linear regression of the total sample (n=45) involving both treated (n=36) and controls (n=9), where hippuric acid values versus quinic acid values collected from urine at the same time in the same subject gave y=0.0157x+0.1839, r=0.60, p<0.001.

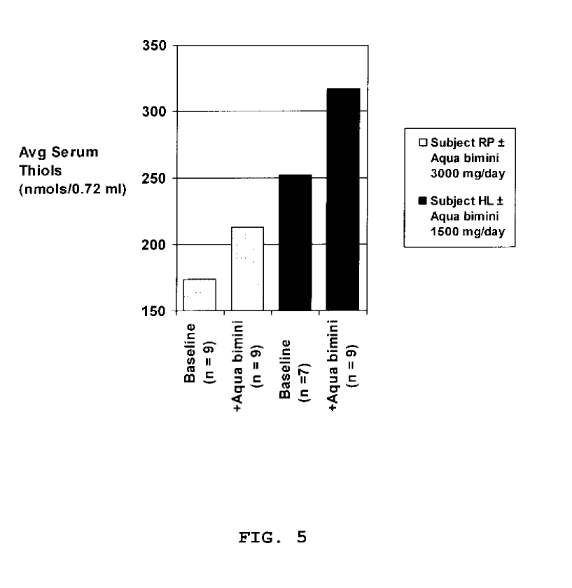

FIG. 6. Illustrates the individual baseline serum protein thiol(s) estimations determined on subject HL over 83 days before oral administration of Aqua Bimini™ (quinic acid ammonium chelate) at 3000 mg/day for 36 days, and then followed-up with no treatment for 8 months.

FIG. 7. Illustrates the individual baseline serum protein thiol(s) estimations determined on Subject HL over 83 days before oral administration of Aqua Bimini™ (quinic acid ammonium chelate) at 1500 mg/day for 36 days, and then followed-up with no treatment for 8 months.

FIG. 8. Illustrates orally administered Aqua Bimini™ (quinic acid ammonium chelate) for 37 days followed by a dry-out period of an additional 32 days enhanced the in vivo antioxidant status of trial participants when assessed by the anti-oxidant parameter of serum protein thiols. Both doses of Aqua Bimini™ (quinic acid ammonium chelate) were statistically different from their corresponding baseline values analyzed by students t-test (p<0.001).

FIG. 9. Illustrates urinary profile of subject RP treated with 3000 mg/day Aqua Bimini™ (quinic acid ammonium chelate) for 36 days and then followed for an additional 8 months with no further treatment. Urinary tryptophan and nicotinamide levels (μM) were estimated on the sampling dates indicated.

FIG. 10. Illustrates urinary profile of subject HL treated with 1500 mg/day Aqua Bimini™ for 36 days and then followed for an additional 8 months with no further treatment. Urinary tryptophan and nicotinamide levels (μM) were estimated on the sampling dates indicated.

FIG. 11. Illustrates the in vivo animal studies estimation of blood cells and serum tryptophan after 21 days treatment with either 4 mg/ml C-Med-100® or 2 mg/ml quinic acid). The levels of hippuric acid (<0.02 μg/ml) and quinic acid (<2 mg/ml) were also simultaneously determined, but found to be below HPLC detection when quinic acid was dosed at 1 mg/ml in drinking water of mice or about 500 mg/kg.

### DETAILED DESCRIPTION OF THE INVENTION

The present invention provides for increasing the levels of aromatic amino acids such as tryptophan, tyrosine and phenylalanine or the vitamin precursor nicotinamide in serum and/or urine and/or biologic tissues in a mammal, by administering an effective amount of their precursors in the shikimate pathway. Raised levels of tryptophan and/or nicotinamide levels in serum and/or biologically responsive tissues in the mammal body, which in turn can raise the serotonin, dopamine, NAD or enhance DNA repair levels in the mammal body.

In higher animals, aromatic amino acids are generally derived from dietary sources such as animal and vegetable proteins. The inventor has discovered that aromatic amino acids can synthesized by the gastrointestinal bacterial flora through the pathway, commonly known as shikimate (or shikimic acid) pathway. As an example, gastrointestinal bacteria such as E-coli are capable of converting erythrose 4-phosphate to shikimate, chorismate and finally phenylalanine, tyrosine or tryptophan.

In an embodiment precursors to aromatic amino acids in the gastrointestinal tract (GI) tract facilitate the shikimate pathway to convert said precursors to aromatic amino acids such as tryptophan, tyrosine, phenylalanine, or other essential vitamins such as nicotinamide. It has been established previously that nicotinamide is synthesized via tryptophan. (See Satyanarayana, U, Narasing a, U., Rao, B. S., Effect of diet restriction on some key enzymes tryptophan-NAD pathway in rats, Jour. Nutrition 107 (12): 2213-2218, 1977; Satyanarayana, U., Rao, B. S., Effect of dietary protein level on some key enzymes of the tryptophan-NAD pathway, Brit. Jour. Nutr. 38(1): 39-45, 1977). Aromatic amino acids and vitamins mediate cell processes controlled by serotonin, dopamine, and they also play a significant role in DNA repair. For example, NAD is an important co-factor in more than 500 biochemical reactions in the body including the DNA repair process (Okamoto, H., Ishikawa, A., Yoshitake, Y. et al., Diurnal variations in human urinary excretion of nicotinamide catabolites: effects of stress on the metabolism of nicotinamide. Amer. Jour. Clinical Nutrition 77(2): 406-410, 2003).

A key biochemical event, not disclosed previously, is that at the efficacious dose of 25 mg/kg quinic acid chelate in humans, there was no quinic acid found in human serum at the detection limit of the HPLC method (1 mg/kg; i.e. only 4% of the efficacious dose). The inventors, thus, not being bound by any theory believe that quinic acid itself could not be the direct-acting bioactive ingredient.

As used herein the term “chelate” encompass a ratio of free acid to ion (e.g., ammonium ion or any other pharmaceutically acceptable ion) wherein the represented ion in the ratio is not a whole number, e.g., 1:1.2, 1:1.3, 1:1.4, 1:1.5 and 1:1.6, as well as values in between, e.g., 1:1.54 (quinic acid saturated with ammonium ions). However, depending upon the conditions, particularly the pH of the solution, the chelate ratios vary. Although as discussed herein close to a 1:1.54 ratio is preferred quinic acid chelate compositions of the present invention are by no means limited to those with a 1:1.54 ratio of quinic acid to ammonium ion.

Determination of salts and chelates of some naturally occurring polyhydroxylated and polycarboxylated organic acids are provided in Table 1. Experimental molar ratios were calculated from neutralization to pH=7.5 of free protonated organic acid (H⁺) with sodium, potassium or ammonium hydroxides.

TABLE 1

| | Molar ratio | Experimental | |
|---|---|---|---|
| | of salt | molar ratio | Determination |
| | theoretical | of molecular | of structure |
| | carboxy(−)/ | equilibrium | existing in |
| Organic acid moiety | cation(+) | in water | water |
| Quinic acid sodium | 1:1 | 1:1 | salt |
| salt | | | |
| Quinic acid | 1:1 | 1:1.30 | chelate |
| potassium salt | | | |
| Quinic acid | 1:1 | 1:1.54 | chelate |
| ammonium salt | | | |
| Hydroxy citric acid | 1:3 | 1:1.85 | Chelate |
| ammonium salt | | | |
| Organic acid moiety | Molar ratio | Experimental | Determination |
| | of salt | molar ratio | of structure |
| | theoretical | of molecular | existing in |
| | carboxy(−)/ | equilibrium | water |
| | cation(+) | in water | |

Quinic acid in the free acid (H+) form or obtained from hydrolyzed quinic acid esters (to release quinic acid in situ) is treated with excess ammonia (10% ammonia, for example, for 2 hours, for example) to generate quinic acid ammonium chelate described and characterized herein as efficacious in vivo.

The present invention is also directed to processes to convert substantially all forms of quinic acid in plant material into a quinic acid chelate, particularly quinic acid ammonium chelate, and to the related production of improved medicinal compositions which exhibit increased biological efficacy and decrease toxicity. Particularly preferred compositions of the present invention comprise a substantial amount or at least an effective amount of least one quinic acid chelate to exhibit at least one biological activity property described herein.

Substantial amount, as used herein, refers to compositions wherein a quinic acid chelate represents more than 5% of all forms of quinic acid present in the composition, preferably more than 15%, and most preferably more than 25%. Preferably, at least one quinic acid chelate is the major form of quinic acid that is present in the composition. Major form, as used herein, refers to compositions wherein a quinic acid chelate represents more than 50% of all forms of quinic acid present in the composition, preferably more than 60%, and most preferably more than 70%.

Compositions are preferred wherein at least one quinic acid chelate is the substantially major form of quinic acid that is present in the composition. Substantially majority form, as used herein, refers to compositions wherein a quinic acid chelate represents more than 50% of all forms of quinic acid present in the composition, preferably more than 60%, and most preferably more than 70%.

Compositions are preferred, for example, wherein quinic acid ammonium chelate is the substantially major form of quinic acid that is present in the composition or wherein quinic acid ammonium chelate is the only form of quinic acid that is substantially present in the composition. Quinic acid ammonium chelate as the only form that is substantially present, as used herein, refers to compositions wherein a quinic acid chelate represents more than 90% of all forms of quinic acid present in the composition, preferably more than 95%, and most preferably more than 99%. Compositions are described herein, for example, wherein quinic acid ammonium chelate is present as substantially the only form of quinic acid in the composition.

In an embodiment method the precursors to the aromatic amino acids such as quinic acid, shikimic acid, and/or chorismate, their chelates thereof, is/are typically administered in an amount to increase serum trytophan and nicotinamide concentrations in a mammal by at least 30% of non-supplemented (baseline) levels or to levels that significantly enhance production of serotonin, dopamine and NAD, or enhance DNA repair above existing background levels. Aromatic amino acids mediate cell processes controlled by serotonin, dopamine, NAD and DNA repair that can in turn provide potent anti-oxidant effects. For example, NAD is an important co-factor in more than 500 biochemical reactions in the body including but not limited to DNA repair (Okamoto, H., Ishikawa, A., Yoshitake, Y., et al., Diurnal variations in human urinary excretion of nicotinamide catabolites: effects of stress on the metabolism of nicotinamide. Amer. Jour. Clinical Nutrition 77(2): 406-410, 2003).

Typically, precursors to aromatic amino acids including the chelates of quinic acid, shikimic acid, and/or chorismate are administered in a systemic dose of 500 mg/day to 5000 mg/day or about 6.7 to 66.7 mg/kg in humans, delivered in drinking water, capsules, tablets. Preferred dosage forms are orally in administered in liquid from (e.g. drinking water) or orally administered in dry form as capsules or tablets or such formulations that are readily combinable with other foods or beverages

The invention is also further directed to a process for the production of an isolated medicinal composition comprising an effective amount of a quinic acid chelate, preferably an ammonium chelate in about a 1:1.54 ratio of quinic acid to ammonium ion.

The precursor for an aromatic amino acid has the following formula A-(X)ₙ where X is selected from the group consisting of any monovalent, divalent, trivalent anions that can form salts or hydroxides or amines, and “n” is an integer or a fraction, wherein 1<n=10. A is as described previously herein having the structures (I)-(V), anions, esters thereof, wherein R is H or C₁-C₃ alkyl. Preferred are when R is H. The moiety X for example can be ammonium chloride or ammonium hydroxide, potassium chloride or potassium hydroxide, magnesium chloride or magnesium hydroxide, zinc chloride or zinc hydroxide, calcium chloride or calcium hydroxide, or any pharmaceutically acceptable salt thereof. Examples of such moieties include but not limited to quinic acid ammonium chelate, quinic acid potassium chelate, quinic acid zinc salt chelate, quinic acid lithium salt chelate, quinic acid calcium chelate.

In another embodiment the moiety X can be an amino acid such as histidine salt, lysine salt. Examples of such moieties include quinic acid histidine salt/chelate and quinic acid lysine salt/chelate. In yet another embodiment, the moiety X can be an amine such as aminoethylethanol amine, ethanolamine, diethanolamine, triethanolamine, isopropanol amine, or chelating agents such as EDTA or DETA. Any pharmaceutically acceptable amine or alkanolamine and/or chelating agents known to persons skilled in the art can be used.

In an embodiment the precursor for the aromatic amino acid could be quinic acid derivatives which could lead to the structures (I)-(V). In another embodiment the precursors could be racemic mixtures or enantiomers thereof.

Serum thiols are used in vivo surrogate estimate of oxidative stress and DNA repair capacity and is usually estimated as total serum protein thiols as disclosed in U.S. Pat. No. 5,925,571 to Pero. We have used 80% ammonium sulfate precipitated sub-fraction of serum thiols and shown that this estimate was satisfactory for the purpose of showing the antioxidant effect of Aqua Bimini™ (a quinic acid ammonium chelate). (See Banne, A., Amiri, A., Pero, R. W., Reduced Level of Serum Thiols in Patients with a Diagnosis of Active Disease. JAAM 6(4): 325-32, 2004; Pero, R. W., Giampapa, V., Vojdani, A., Comparison of a broad spectrum anti-aging nutritional supplement with and without the addition of a DNA repair enhancing cat's claw extract. J. Anti-aging Med. 5(2): 345-353, 2002; Pero, R. W., Hoppe, C., Sheng, Y., Serum thiols as a surrogate estimate of DNA repair correlates to mammalian life span, Jour Anti-Aging Med 3(3): 241-249, 2000; Pero, R. W., Amiri, A., Welther, M., Rich, M., Formulation and clinical evaluation of combining DNA repair and immune enhancing nutritional supplements. Phytomedicine 12(4): 255, 2005)). Quinic acid chelates are described in WO 2006/101922 to Pero, which is incorporated herein by reference in its entirety.

In an embodiment obtaining the ratio of urinary levels of nicotinamide or tryptophan or their combination (i.e. nicotinamide+tryptophan) to serum protein thiols allows one of ordinary skill in the art to predict the effectiveness of an anti-oxidant therapy and other health benefits from such intervention (e.g. quinic acid ammonium chelate that decreases in serum thiol(s)/tryptophan or nicotinamide ratios) or the presence of disease states (i.e. increases in serum thiol(s)/tryptophan or nicotinamide ratios).

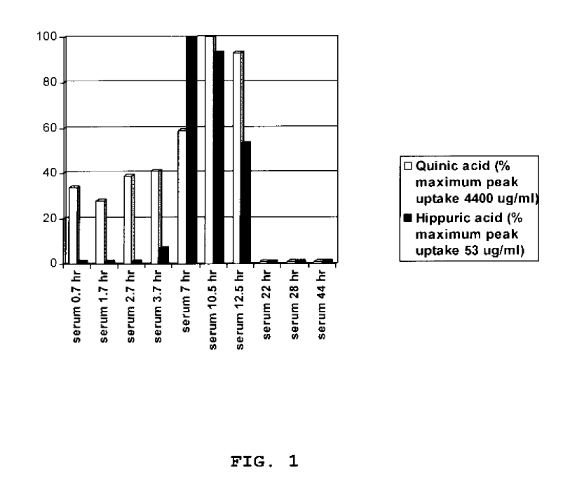

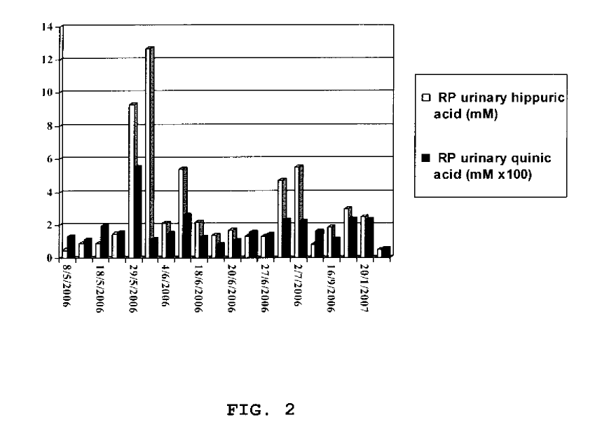

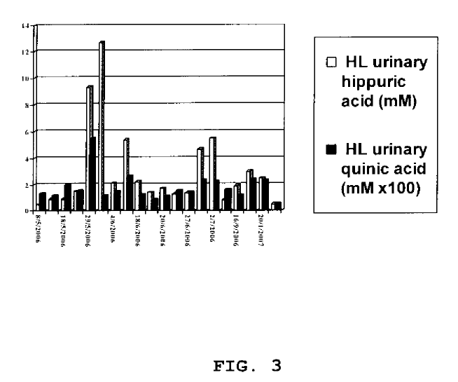

The inventor determines herein that quinic acid does not by itself provide beneficial effects in vivo because it is not taken into peripheral circulation (See FIG. 1). Rather, it is the precursor form (See FIGS. 2-3) to the shikimate pathway that provides the desired clinical activity.

The inventor of the instant application have now discovered that precursors of aromatic amino acids comprising quinic acid derivatives, shikimic acid, and/or chorismate are active as indicated by their effect on serum levels of tryptophan or nicotinamide. The inventor while not being bound by any theory believe that quinic acid chelates exhibit enhanced bioactivity and attributes to the elevated levels of tryptophan resulting from introduction of aromatic amino acid precursors into the shikimate pathway. It is believed that quinic acid, itself, is not the bioactive compound in vivo because there is no direct evidence of its presence in peripheral circulation to mediate any efficacious effect.

The aromatic amino acid precursor is typically administered in an amount to increase serum tryptophan and nicotinamide concentrations in a mammal by at least 10% of non-supplemented (baseline) levels, preferably to levels of at least 30% or greater. The aromatic amino acid precursors are preferably administered to humans in a systemic dose up to 5000 mg/day, preferably 1000 mg/day to 5000 mg/day or about 14.2 to 66.7 mg/kg, delivered in drinking water, capsules, tablets, subcutaneous injection, intraperiteonal injection, intravenous injection or topically to skin. In an embodiment the quinic acid chelate (present as Aqua Bimini™ is administered).

Preferred dosage forms are orally in administered in liquid from (e.g. drinking water) or orally administered in dry form as capsules or tablets or in combination with meal.

Embodiment methods involve determining in vivo oxidative stress levels in a patient by determining the ratio of either urine tryptophan or nicotinamide or a combination thereof/serum thiols of the patient, after the administration of an aromatic amine precursor to the patient.

Another embodiment method involves obtaining levels of the individual's serum protein thiol(s) and levels of urinary tryptophan, nicotinamide, hippuric acid, or all, after the end of the treatment cycle, and establishing a value for the ratio of urinary tryptophan, nicotinamide or combination thereof/serum protein thiol(s) for the individual, after said treatment cycle.

In an embodiment method baseline and the predetermined normalized levels of a population afflicted from said disease is compared to establish the efficacy of the antioxidant therapy. Efficiency of the antioxidant therapy is determined by at least about a 25% increase, a 30% increase, a 50% increase, a 75% increase, a 100% increase, a 150% increase, preferably greater than about a 50% increase.

An important biochemical event not previously understood or disclosed is that at the efficacious dose of 25 mg/kg quinic acid chelate in humans, there was no quinic acid found in human serum at the detection limit of the HPLC method (1 mg/kg; i.e. only 4% of the efficacious dose). Hence, quinic acid itself could not be the direct-acting bioactive ingredient.

One of ordinary skill in the art would appreciate that oral administration of quinic acid formulations of the instant application does not cause a significant elevation of quinic acid levels in humans. Accordingly, the ordinary skill in the art would recognize that the mechanism of action of the precursors of aromatic amino acids for production of tryptophan and nicotinamide is via the shikimate pathway of the GI tract microflora. The prior art provides only that quinic acid at about 250 mg/kg in mice and rats had efficacious effects. However, it incorrectly concluded that the effects were due to quinic acid, and not such metabolites as tryptophan or nicotinamide (Sheng, Y., Akesson, C., Holmgren, K., Bryngelsson, C., Giampapa, V., Pero, R. W., An active ingredient of Cat's Claw water extracts: Identification and efficacy of quinic acid. Journal of Ethanopharmacology 96(3): 577-584; 2005). Contrary to the teaching of the prior art the inventor determined that the efficacious effects were in the dose ranges of 500 to 5000 mg/day, preferably 1500 to 5000 mg/day, or 6.7 to 66.7 mg/kg in humans.

The inventor has found that the oral administration of precursors of aromatic amino acids to warm blooded animals (i.e. mammals including humans) may be used to raise the level of tryptophan and/or nicotinamide levels in serum and/or biologic responsive tissues in the mammal body. It is believed that clinical effects may also be achieved by co-administration, of quinic acid complexes, and other anti-oxidants such as shikimic acid/shikimate and chorismate, vitamins, bioflavonoids, biophenols. The methods of the instant invention may be used to raise the levels of aromatic amino acids such as tyrosine and phenylalanine in serum urine and/or other biologic tissues in a mammalian body.

Another advantage of the oral administration of the instant precursors to the aromatic acids to increase tryptophan and nicotinamide levels is that this form of supplementation is regulated by normal gastrointestinal tract metabolism, avoiding possible toxic effects associated with direct tryptophan supplementation.

Precursors to the aromatic amino acids may be obtained by commercially purifying chichona bark, for e.g. obtained from Sigma or Acros. At least one feature of the instant invention is directed to foods or additives that contains or forms the precursors to the aromatic amino acid.

Quinic acid-containing functional foods may also serve as good sources for quinic acid because such products are not chemically synthesized. Yet, in at least one aspect of the instant invention, the instant compounds may be added in their natural, purified, isolated or synthetic forms to such food products in order to optimize the content of the desired aromatic amino acid precursors in said products. Food sources that have a level of higher than 0.5% quinic acid content and can be used include but are not limited to Cat's Claw, prune, kiwi, sea buckthorn, coffee, cranberry, lingonberry, blueberry, wortleberry, red/yellow tamarillo, and sultana. Such sources are more preferred because they may not require any additional chemical modification. Food sources having quinic acid content <0.5% could also be used. Examples of food additive quinic acid sources in this category are quince, sunflower, nectarine, peach, pear, plum, honey, black currant, medlar, apricot, asparagus, mushroom and green olive.

Typically a precursor to an aromatic amino acid such as a quinic acid chelate, is administered in a systemic dose of 500 mg/day to 5000 mg/day or about 6.7 to 66.7 mg/kg in humans, delivered in drinking water, capsules, tablets, subcutaneous injection, intraperiteonal injection, intravenous injection or topically to skin. Preferred dosage forms are orally in administered in liquid from (e.g. drinking water) or orally administered in dry form as capsules or tablets or combined with a food product.

Examples of health disorders that could be treated by elevating tryptophan and nicotinamide to mediate health effects include those modulated by the serotonin, dopamine and nicotinamide/NAD receptors. For example, water extracts of Cat's Claw prevented or controlled ulcerative colitis (inflammatory responses), osteoarthritis/joint pain, tumor cell growth, weight gain, ozone injury, DNA damage/cell death, chemotherapeutic-induced leucopenia and dementia/Alzheimer's. See Sandoval-Chacon, M., Thompson, J. H., Zhang, X. J., Liu, X., Mannick, E. E., Sadowicka, H., Charbonet, R. M., Clark, D. A., Miller, M. J., Anti-inflammatory actions of Cat's Claw: the role of NF-kappa B. Alimentary Pharmacological Therapy 12: 1279-1289, 1998; Piscoya, J., Rodriguez, Z., Bustamente, S. A., Okuhama, N. N., Miller, M. J., Efficacy and safety of freeze dried cat's claw in osteoarthritis of the knee: mechanisms of action of the species Uncaria guianensis. Inflammation Res 50: 442-448, 2001. Castillo and Snow U.S. Pat. No. 6,346,280 issued February 2002. Persistent response to pneumococcal vaccine in individuals supplemented with a novel water soluble extract of Uncaria tomentosa, C-Med-100®. Phytomedicine 8(4): 267-274, 2001; Sheng, Y., Bryngelsson, C., Pero, R. W., Enhanced DNA repair, immune function and reduced toxicity of C-MED-100®, a novel aqueous extract from Uncaria tomentosa. Journal of Ethnopharmacology 69:115-126, 2000; Sheng, Y., Li, L., Holmgren, K., Pero, R. W., DNA repair enhancement of aqueous extracts of Uncaria Tomentosa in a human volunteer study. Phytomedicine 8(4): 275-282, 2001; Akesson, C., Pero, R. W., Ivars, F., C-Med-100®, a hot water extract of Uncaria tomentosa, prolongs leukocyte survival in vivo. Phytomedicine 10: 25-33, 2003; Akesson, C., Lindgren, H., Pero, R. W., Leanderson, T., Ivars, F., An extract of Uncaria Tomentosa inhibiting cell division and NF—B activity without inducing cell death, International Immunopharmacology 3: 1889-1900, 2003. All of which are hereby incorporated by reference.

In addition, psychiatric and neurodegenerative disorders, depression, mood and pellagra (Vitamin B3 deficiency) may be successfully treated with aromatic amino acid precursors at doses sufficient to increase tryptophan and/or nicotinamide, and/or serotonin levels and improve the symptoms of such conditions.

Among other ingredients Cat's Claw extract can contain various antioxidants, flavonoids and polyphenols and other compounds including quinic acid, ajmalicine, akuammigine, campesterol, catechin, carboxyl alkyl esters, chlorogenic acid, cinchonain, corynantheine, corynoxeine, daucosterol, epicatechin, harman, hirsuteine, hirsutine, iso-pteropodine, loganic acid, lyaloside, mitraphylline, oleanolic acid, palmitoleic acid, procyanidins, pteropodine quinovic acid glycosides, rhynchophylline, rutin, sitosterols, speciophylline, stigmasterol, strictosidines, uncarines, and vaccenic acid.

In an embodiment the formulation containing precursor to the aromatic amino acids can comprise a secondary natural, purified, isolated or synthetic antioxidant selected from the group consisting of quecrcitin, rutin, chrysin, myricetin, genisten, hesperidine, naringin, and mixtures thereof.

In an embodiment the formulation containing precursor to the aromatic amino acids can also comprise a secondary antioxidant selected from the group consisting of coenzyme Q, pyruvate, coenzyme A, ubiquinol, NADH, NAD, NADP, NADPH, adenine, adenosine, niacin, nicotinamide, campesterol, catechin, chlorogenic acid, cinchonain, corynantheine, corynoxeine, daucosterol, epicatechin, harman, hirsuteine, hirsutine, iso-pteropodine, loganic acid, lyaloside, mitraphylline, oleanolic acid, palmitoleic acid, procyanidins, pteropodine, quinovic acid glycosides, rhynchophylline, sitosterols, speciophylline, stigmasterol, strictosidines, uncarines, and vaccenic acid.

In a preferred embodiment the formulation containing precursor to the aromatic amino acids can comprise a secondary anti-oxidant compound selected from the group consisting of nicotinamide, niacin, NAD, NADP, flavonoids, cinnamic acid or derivatives thereof, ascorbic acid or derivatives thereof, tocopheral or derivatives thereof, and vitamin A or D or derivatives thereof.

The formulation containing the precursors to the aromatic amino acids can further contain a pharmaceutically acceptable vehicle which can further comprise optional therapeutic ingredients. These optional ingredients are selected from the group consisting of anti-neoplastic agents, anti-infectives, anti-depressants, mood-enhancing agents, and anti-inflammatory agents.

In one embodiment the anti-neoplastic agents are selected from the group consisting of fluorinated pyrimidines, cytidine analogues, purine antimetabolites, plant alkaloids, alkylating agents, anthracene derivatives, antitumor antibiotics, metal complexes, anti-aminophospholipid antibodies, anti-angiogenic agents, and radiotherapeutics.

In another embodiment the optional ingredient is an anti-infective selected from the group consisting of sulfonamides, penicillins, cephalosporins, aminoglycosides, protein synthesis inhibitors, antifungals, antiviral agents, anti-tuberclosis agents.

In another embodiment the optional ingredient is an anti-depressant selected from the group consisting of tricyclic anti-depressants, and serotonin reuptake inhibitors.

In yet another embodiment the optional ingredient maybe an anti-inflammatory agent selected from the group consisting of steroidal anti-inflammatory agents, and non-steroidal anti-inflammatory agents.

In another embodiment a preferred process for the production of an isolated medicinal composition is described wherein the process comprises providing an effective amount of a quinic acid chelate comprising, combining substantially pure quinic acid, with ammonium hydroxide in an aqueous solution sufficient to reach a pH from about 6.9 to about 7.6, to yield an ammonium chelate of quinic acid wherein a ratio of quinic acid to ammonium ion is about 1:1.54. Processes are preferred wherein a solution of ammonium hydroxide, from about 1% and about 10% in concentration, is added to an aqueous solution of quinic acid which comprises from about 5 g to about 30 g quinic acid per 100 ml, in a sufficient amount for the solution to reach a pH from about 7.4 and to about 7.6 within a time period from about 15 minutes to about four hours.

Embodiment compositions described herein are produced, for example, by converting substantially pure D-Quinic acid to the ammonium chelate in about a 1:1.6 molar ratio in an aqueous medium using ammonium hydroxide within the range of about pH 7 to about pH 7.5. A pH of about 7.5 is preferred. However, quinic acid ammonium chelates described herein may be produced, for example, at pH 6.9, 7, 7.1, 7.2, 7.3, 7.4, 7.5, and 7.6, and at all pH values in between. Other methods of making a quinic acid chelate may be employed by those of ordinary skill in the art without departing from the spirit and scope of the articulated methods herein.

Embodiment pharmaceutical or nutraceutical compositions comprise a significant and effective amount of the ammonium chelate of quinic acid. In an embodiment Qunimax™ a ammonia-treated quinic acid forms a substantially pure ammonium chelate in about a 1:1.6 (actually 1:1.54) molar ratio at a physiological pH can be used. Preferably, Quinmax™ is substantially pure quinic acid neutralized with aqueous ammonia to pH=7.5. Materials and Procedures

(a) Sample Collection and Preparation

Blood samples were collected by venal puncture using vacutainers (red top, 10 ml) usually in the morning (a.m.) after 12 hours of fasting. Serum was separated from the blood clot after setting 2 hr on the bench top before centrifugation for 10 min at 1500×g. Sera prepared in this manner were stored at +4° C. until biochemical analysis usually within 1-30 days of collection. Urine samples were collected as a random 50 ml sample in the A.M. during the treatment and follow-up (after treatment for 8 months) periods. Urine was spun at 5000×g for 15 min and the supernatant stored at +4° C. until analyzed within 1 month of collection. After the first analysis all samples were further stored at −20° C. and used for repeat determinations over time.

(b) Trial Design

Trial design for the clinical evaluation of Aqua Bimini™ (quinic acid ammonium chelate) was carried out at Lund University beginning Apr. 28, 2006. Two Subjects identity code protected as HL and RP received Aqua Bimini™ at 1500 mg/day and 3000 mg/day, respectively for 4 consecutive weeks (36 days). Repeat serum and urine samples were collected throughout the trial period and then pooled into groups for analysis as 6 weeks before (baseline), 4 weeks of intervention (treatment, 36 days) and 8 months dry-out (no treatment).

A reference group was established for comparison to normal unsupplemented individuals. It consisted of 9 individuals who had never taken quinic acid as a supplement; there were 6 males, 3 females aged 12 to 86 years of age. None smoked but 6 of 9 were taking micronutrient supplements before and during the evaluation period. This non-clinical pharmacokinetic research program was conducted according to the guidelines of the Declaration of Helsinki for humans. Moreover, informed consent was obtained from all participants that included individual permission to obtain blood and urine samples only for use in this study, and with institutional review approval.

The unexposed (controls) and nutrient supplemented levels in urine of quinic acid, hippuric acid, tryptophan or nicotinamide are presented in Table 3. The mean urinary values for each group including both supplemented and unexposed controls were compared by t-test statistics to assess efficacy and metabolism of Aqua Biminin™.

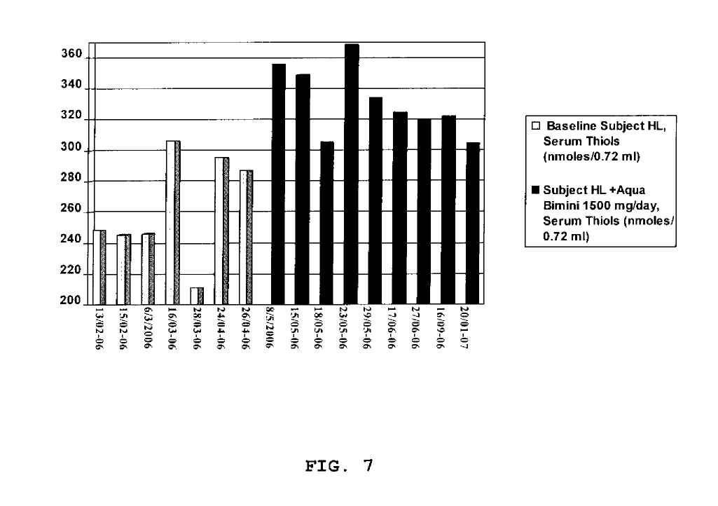

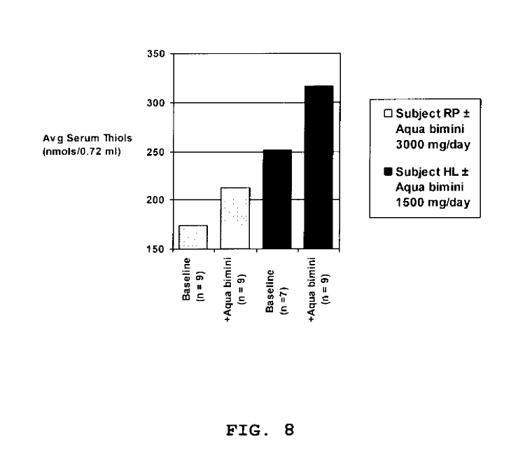

In addition, this baseline-controlled trial involving a non-clinical pharmacokinetic evaluation also compared serum samples before supplementation (i.e. baseline) to during supplementation (4-5 weeks) which were compared against the dry-out values (immediately after with no treatment for 8 months) for the same individual. The total individual serum protein thiol values can be viewed and evaluated later on as shown in FIGS. 7 and 8. In such a manner, inter-individual variation in these biochemical parameters were randomized out of the protocol, because individual variation in the antioxidant test parameter (serum protein thiols) had been taken out of the design by controlling differences in individual baseline values before start of treatment.

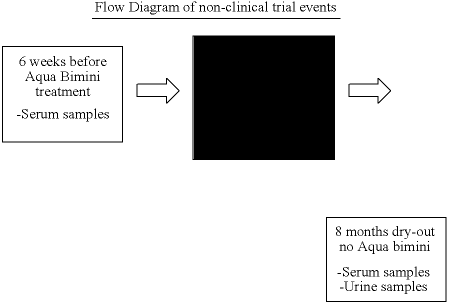

(c) Estimation of Serum Protein Thiols as an In Vivo Anti-Oxidant Indicator

The following detailed description was designed to standardize the estimation of the in vivo level of serum protein thiols that have been used previously to estimate DNA repair capacity. When a blood sample is collected and the serum isolated, then the level of serum protein thiols is an estimate of the concentration between thiols and disulfides existing in protein structures that are in turn equilibrated to the existing oxidant environment in vivo. Thus, serum protein thiol analyses are an in vivo estimate of the antioxidant status of an individual that is in turn regulated by DNA repair.

(d) Establishment of a Standard Thiol-Sensitive Curve for Quantitative Measurement of Protein Thiols

A stock solution of the colorimetric agent 5,5′-dithio-bis-(2-nitrobenzoic acid (DTNB), was prepared using: 9.5 mg/ml solid DTNB, 0.1 M K₂HPO₄, and 17.5 mM EDTA. pH was adjusted to 7.5 and then diluted to desired volume. Working DTNB solution was prepared by diluting DTNB stock solution 1:50 with saline. Solutions of L-cysteine were prepared and a standard curve was constructed desirably be in the 0-100 μM range. Stock solutions were diluted as required and absorbance was measured using 96-well flat-bottom microtiter plate. 50 μL-cysteine solution with 200 μl working DTNB solution was placed per well. Two to three replicates per concentration were made.

A DTNB blank (50 μl saline+200 μl DTNB) was made. Absorbance values were made at 412 nm with a microtiter plate scanner spectrophotometer. The average of every concentration was calculated and DTNB blank was subtracted from every value. Using a plot of concentration (x-axis) vs. absorbance (y-axis) by comparison with standard cysteine solutions the cysteine concentration was determined.

(e) Measurement of Serum Protein Thiols

A saturated ammonium sulfate solution was prepared. Using a 200 μl serum for every sample, the serum was precipitated with 800 μl saturated ammonium sulfate. Samples were centrifuged in 1.5 ml vials at 12,000 G for 15 minutes at room temperature. When the bottom of the vial contained a solid pellet the supernatant was discarded (approx 800 μl) without disturbing the bottom. Then the pellet was re-suspended in 600 μl saline. Transparent 96-well flat-bottom microtiter plate was used in analysis. One replicate was prepared as follows: (i) A 50 μl aliquot of serum with 200 μl working DTNB in one well, and (ii) a serum background by putting a 50 μl aliquot with 200 μl saline in another well. This was repeated three times for every sample. A DTNB blank was made (50 μl working DTNB+200 μl saline) and a saline blank (250 μl saline) was also made in three replicates. Absorbance in microtiter plate scanner set at 412 nm was measured making sure that bubbles in the well are avoided.

(f) Estimate of Serum Protein Thiols from the Cysteine Standard Curve

The average was calculated for the DTNB blank and the saline blank. For every replicate, the DTNB blank was subtracted from every sample value (corrected sample). Also the saline blank was subtracted from every serum value (corrected serum). See example as outlined in Table 2 below to illustrate the calculations.

Then the corrected serum value was subtracted from the corrected sample value. This value was the final serum thiol value expressed as absorbance at 410 nm. This average value was calculated for all of the replicates as outlined below in the example provided are provided in Table 2.

TABLE 2

| | Column # | | | | | | |
|---|---|---|---|---|---|---|---|
| | 1 | 2 | 3 | 4 | 5 | 6 | 7 |
| | Sample | DTNB blank | Serum | Saline | Corrected | Corrected | Final |
| Name (rep) | A₄₁₂ | A₄₁₂ | A₄₁₀ | blank A₄₁₂ | sample (1-2) | serum (3-4) | (5-6) |
| A (1) | 0.3926 | 0.1792 | 0.0778 | 0.0425 | 0.2134 | 0.0353 | 0.1781 |
| A (2) | 0.3979 | 0.1792 | 0.0795 | 0.0425 | 0.2187 | 0.0370 | 0.1817 |
| A (3) | 0.3974 | 0.1792 | 0.0779 | 0.0425 | 0.2182 | 0.0354 | 0.1828 |
| Average | | | | | | | 0.1809 |
| B (1) | 0.3687 | 0.1792 | 0.0955 | 0.0425 | 0.1895 | 0.0530 | 0.1365 |
| B (2) | 0.3705 | 0.1792 | 0.0980 | 0.0425 | 0.1913 | 0.0555 | 0.1358 |
| B (3) | 0.3690 | 0.1792 | 0.0961 | 0.0425 | 0.1898 | 0.0536 | 0.1362 |
| Average | | | | | | | 0.1362 |

The average value shown in Table 2 was entered into the standard curve for determination of corresponding cysteine molar concentration. Thus, the value is the molar concentration of serum thiols expressed as cysteine molar equivalents. After the serum was precipitated and spun in the centrifuge, the pellet was re-suspended in 600 μl saline. With the volume increase of the pellet, the final volume was 800 μl; 200 μl pure serum was separated in the beginning. This was therefore a 4 fold dilution. The sampled serum was then diluted 5 times when put in the well (50 μl in 250 μl).

Altogether this was a 20 fold dilution (4×5=20). Thus, the thiol value read off the standard curve should be multiplied with a factor of 20. In addition, in order to make our data comparable with documented and archived data it was needed to multiply with a factor of 0.722. This was due to previously used factors. Thiols were hereby measured in nmoles/0.72 ml.

g) General Conditions of High Pressure Liquid Chromatography (HPLC)

HPLC analysis of hippuric acid, quinic acid, nicotinamide, and tryptophan were carried out using a Perkin Elmer 200 LC pump equipped with a UV detector 785 A. The identification of each compound being evaluated by HPLC was also confirmed independently by thin layer chromatography (TLC) analyses. The columns were either C18 150×4.6 mm or C18 80×4.6 mm Perkin Elmer-Brownlee (Pecosphere part no. 0258-0196 or 0258-0166) or in tandem with a Perkin Elmer C18 30×4.6 mm Brownlee precolumn. The mobile phase was pumped through the column at 1 ml/min with 1500-5000 psi. The UV detector was set at the wavelength of 200 nm-230 nm depending on the compound being detected.

An injection loop of 20 μl was used in all experiments. The data were stored and reprocessed using PE Nelson Turbochrom 4 (S270-0052). C18 columns were regenerated with 30 min washes at 1 ml/min using the following sequence of solvents: acetonitrile:methanol (30:70, v/v/), 100% methanol, methanol:water (50:50), methanol: 0.2% TFA, and 100% 0.2% TFA. In all cases quantitative estimates were based on peak height calculations using analytical grade purity of commercially available standard compounds. These general conditions applied to all HPLC analyses performed in this study.

(h) HPLC Sample and Standard Curve Conditions for Quantifying Quinic Acid in Serum

Serum samples of 0.2 and 30 ml were collected from blood, and precipitated with either ethanol or trichloroacetic acid (TCA). After clean-up the supernatants were dried (ethanol precipitated) or used directly. A standard curve was prepared using quinic acid (Sigma) dissolved in distilled water and obeyed the equation y=6012.8x−24698; 20 μl injections of solutions between 0-25 mg/ml were used.

(i) Single High Dose Quinic Acid Time Study

A 65 year old apparently healthy volunteer drank 6 gm of quinic acid ammonium chelate (Quinmax™) dissolved in 300 ml water over a 15 min period, samples were collected, and then about 40 ml of peripheral blood (4-red topped vacutainers) were allowed to clot at room temperature to prepare serum samples by centrifugation. The serum sampling points were 0.7 hr, 1.7 hr, 2.7 hr, 3.7 hr, 10.5 hr 12.5 hr, hr, 28 hr and 44 hr. 30 ml serum samples were precipitated with 50% ethanol, taken to dryness under a stream of air, and redissolved in 1 ml of methanol for simultaneous HPLC analysis of quinic acid and hippuric acid. The data were reported as quinic acid present in 30 ml of serum. The column used for this experiment was a C18 150×4.6 mm. The mobile phase was 0.1% trifluoroacetic (TFA). The UV detector was set at 220 nm and quinic acid eluted with a retention of 2.26 min and hippuric acid at 8.3 min. Sensitivity of the method was about 1/30=0.033 mg quinic acid/ml serum.

(j) Repeat Doses of 1500 Mg and 3000 Mg Per Day Quinic Acid Analyzed in Serum Over a 6 Week Period

Two human volunteers subject HL (20 years) and subject RP (65 years) ingested daily doses of 1500 mg per day or 3000 mg per day and serum samples collected on May 8, May 15, May 18, May 23, May 29, and June 1.

These samples were each analyzed for quinic acid content in 200 μl serum after precipitation with 2 M trichloroacetic acid (TCA). The column used for these experiments was a C18 80×4.6 mm. The mobile phase was 0.2% trifluoroacetic (TFA):methanol 85:15. The UV detector was set at 215 nm and quinic acid eluted with a retention time of 1.40-1.5 min. The sensitivity for detection of this method was about 1 mg quinic acid /ml serum or a detectable dose of 70 mg/kg in humans not 21 to 42 mg/kg as used herein in this study. Thus this protocol could assess if the quinic acid levels in serum would accumulate after repeated daily administration during a period of 4-5 weeks.

(k) HPLC Analysis of Hippuric Acid in Serum

Serum samples were stored at +4° C. and analyzed within 1 month of collection. Serum samples (200 μl) were prepared for analysis by precipitating with 2M TCA (25 μl if 25M TCA). 20 μl injections of the TCA supernatant into the HPLC were made on serum samples collected during 36 days of Aqua Bimini™ supplementation and for 30 days of follow-up (no treatment). Serum were analyzed by HPLC using a C18 80×4.6 mm and a mobile phase of 0.2% trifluoroacetic (TFA):methanol 75:25.

The UV detector was set at 228 nm and hippuric acid eluted with a retention time of 4.0-4.25 min. A standard curve was prepared using hippuric acid (Sigma) dissolved in distilled water had the varying concentrations expressed by the equation y=511x+6616 following 20 μl injections of solutions between 0-0.15 mg/ml and then converted to μM before plotting. This standard curve was adequate for detecting and quantifying the levels of hippuric acid in serum, because of its increased sensitivity which was about 0.01 mg/ml compared to 1 mg/ml for quinic acid by HPLC.

(l) Preliminary Clean-Up of Urine Samples for HPLC Analyses

Urine samples were mixed with 1:2:4 v/v/v of: aqueous urine:95% ethanol:ethyl acetate for 10 min with vigorous shaking. The ethyl acetate layer was allowed to separate with gravity at room temperature, and then it was removed with a pipette and evaporated with an air stream to dryness in a vacuum hood. In this manner 1-2 ml of urine were reconstituted in 0.2 ml water or ethanol which represented a 5-10 fold increase in concentration. Recovery using this method of extraction of metabolites was: quinic acid=53%, hippuric acid=53%, nicotinamide=54%, and tryptophan=66%.

(m) Quinic Acid and Nicotinamide Simultaneous Detection by HPLC in Urine

The urine samples were diluted 1:2:4 v/v/v with ethanol and ethyl acetate, and then the ethyl acetate fraction dried, solubilized in 0.2 ml water and used for HPLC analyses with 20 μl injections directly onto a C18 80×4.6 mm. The mobile phase was 0.2% trifluoroacetic (TFA):methanol:acetonitrile (70:30):water in a ratio 8:8:84 (v/v/v). The UV detector was set at 215 nm, and quinic acid eluted with a retention of 0.97-1.05 min and nicotinamide at 2.6-2.9 min. The detection limit in urine was about 1.5 mg/ml for quinic acid and for nicotinamide it was 0.015 mg/ml. Between 0-17.5 mg/ml quinic acid the dose response was expressed as y=53.52x+33.55, and between 0-0.2 mg/ml nicotinamide the dose response gave the linear regression line of y=4748x−0.1174.

(n) Tryptophan and Hippuric Simultaneous Detection by HPLC in Urine

The urine samples were diluted 1:2:4 v/v/v with ethanol and ethyl acetate, and then the ethyl acetate fraction dried, solubilized in 0.2 ml water and used for HPLC analyses with 20 μl injections directly onto a C18 80×4.6 mm. The mobile phase was 70:30 v/v, 0.2% TFA: 30% methanol. The UV detector was set at 225 nm. Tryptophan eluted after 5.2-5.4 min and hippuric acid after 3.1-3.4 min. Detection limits for tryptophan and hippuric acid in urine were 0.01 mg/ml and 0.02 mg/ml, respectively. Tryptophan within the dose range of 0-0.06 mg/ml yielded a linear regression of y=3557x+3.345, and the dose range of 0-0.15 mg/ml gave a similar linear relationship of y=4592x−72.46 for hippuric acid.

### Example 1

### Characterization of Quinic Acid Metabolism in Human Subjects

A single high dose of 6000 mg was administered orally to a subject, and then 30 ml serum samples were prepared from whole blood samples taken from 0.7 to 44 hours after exposure. As serum samples were precipitated with ethanol and redissolved in 1 ml water, this allowed for a 30-fold increase in concentration of any quinic acid present in each blood sample, rendering the method extremely sensitive to even small amounts of quinic acid. The data are presented in FIG. 1.

(i) Maximum uptake of quinic acid into the serum after 10.5 hr along with the maximum conversion of quinic acid to hippuric acid at about the same time (after about 7 hr in systemic circulation—i.e. in the serum) was observed. Only 4.4 mg/ml of 6000 mg orally administered had accumulated in the blood stream. Only 4.4 mg of the total dose of 6000 mg was taken up into the serum (i.e. 0.073%). This suggests that 99.9% of the quinic acid was either excreted or metabolized within a 44 hr period.

The data in FIG. 1 were confirmed and extended by analyzing serum after 6 weeks of daily administration and follow-up of either 1500 mg or 3000 mg of quinic acid chelate (Aqua Bimini™). Blood samples were taken and serum prepared 6 times from day 10 to day 37, after repeated Aqua Bimini™ oral administration, and these samples were subsequently analyzed for quinic acid. No quinic acid was found in any of the blood serum samples regardless of how long the subjects had been treated with daily quinic acid chelate doses. It was concluded that quinic acid did not accumulate in the blood stream after repeat daily oral administering when analyzed by the established sensitivity of the method even after 27 repeated doses (day 37−day 10=27) of Aqua Bimini™ (i.e. total doses of quinic acid equal to 40,500 mg or 81,000 mg, respectively).

(ii) Since 99.9% of the quinic acid was not found in serum, it was questioned whether it was either being excreted in urine or possibly metabolized to other compounds. Urine samples collected for 4-5 consecutive weeks during oral daily treatment (36 days and during 8 months of follow-up (i.e. no treatment) of Aqua Bimini™ at 3000 mg/day or 1500 mg/day were analyzed for quinic acid (Table 3, FIGS. 2-3). The average levels of quinic acid in urine for the entire evaluation period were found to be about 184 mM±103 (n=19) and 65±49 mM (n=17), respectively. The standard deviations of these mean values indicate large fluctuations of urinary quinic acid with time, nonetheless they still remained elevated after 9 months reflecting metabolic mobilization from some bodily storage depot. Using volume of urine produced per day to be 1.5 liters, detection of about 184 mM after 3000 mg/day oral dose per day quinic acid in urine, or 184 mmole/liter (which converts to 53,039 mg/1.5 liter total urine volume per day) was obtained.

A similar calculation for the oral dose of 1500 mg/day which yielded 65 mM quinic acid in urine gave 18,737 mg/1.5 liter total urine volume per day. These data are summarized in Table 3 and permit the analysis that 56% and 40% of the total dose administered is excreted as unmetabolized quinic acid after the 9 month evaluation period.

Since quinic acid could be detected in urine during the dryout period (i.e. for 8 months after treatment), then it was reasoned that quinic acid might accumulate in the GI tract from repeat dosing, otherwise how could it be found excreted into urine for 8 months of follow-up (no treatment) after any oral supplementation (See Table 3, FIGS. 2-3). Level of quinic acid metabolites found in urine during and immediately after oral administration of either 3000 mg/day or 1500 mg/day Aqua Bimini™ are provided in Table 3.

This points to the very favorable nutritional implications allowing for a sustained production of aromatic amino acids even in the absence of further supplementation of ether quinic acid or essential amino acids or vitamins.

### Example 2

### Characterization of Hippuric Acid as Quinic Acid Metabolism in Human Subjects

Quinic acid was also evaluated as a source of hippuric acid after oral doses of 1500 mg/day and 3000 mg/day of Aqua Bimini™ (quinic acid ammonium chelate) for period of oral administration from April 28 to June 1, and together with samples included from an additional dryout period (no treatment for 8 months) from Jun. 2, 2006 to Jan. 20, 2007.

It is well known that animals including humans can metabolize between 5% to 70% of a dose of quinic acid to benzoic and then to hippuric acid within 1-8 days. Thus hippuric acid has been the only previously known metabolite characterized as originating from quinic acid exposure.

TABLE 3

| | | Tryptophan | Nicotinamide | Hippuric | Quinic acid |
|---|---|---|---|---|---|
| Clinical Treatment | n | (μM) | (μM) | acid (mM) | (mM) |
| Controls (No supplement) | 9 | 10 ± 4 | 23 ± 23 | 1.1 ± 2 | 60 ± 42 |
| RP 3000 mg/day QA (30 | 5 | 13 ± 5 | 21 ± 16 | 2.6 ± 4 | 231 ± 181 |
| days) | | | | | |
| RP 1st 4 months no | 11 | 22 ± 7** | 71 ± 44** | 3.6 ± 3* | 160 ± 57** |
| treatment | | | | | |
| RP 2nd 4 months no | 3 | 19 ± 6 | 71 ± 63 | 2.5 ± 0.6 | 194 ± 70* |
| treatment | | | | | |
| Total RP sample mean | 19 | 19 ± 7** | 58 ± 46** | 3.1 ± 3* | 184 ± 103** |
| HL 1500 mg/day QA (30 | 5 | 18 ± 5** | 163 ± 91** | 1.4 ± 0.8 | 86 ± 52 |
| days) | | | | | |
| HL 1st 4 months no | 6 | 30 ± 8** | 453 ± 139** | 1.5 ± 2 | 84 ± 52 |
| treatment | | | | | |
| HL 2nd 4 months no | 6 | 17 ± 7 | 24 ± 7 | 0.5 ± 0.5 | 30 ± 17 |
| treatment | | | | | |
| Total HL sample mean | 17 | 22 ± 9** | 216 ± 210** | 1.2 ± 1 | 65 ± 49 |

**two way t-test = p < 0.05 versus controls;
*one way t-test = p < 0.05 versus controls

Here we have analyzed both urine and serum for the presence of hippuric acid. In urine mM amounts of hippuric acid were found (Table 3, FIGS. 2-3). For example, urine yielded up to 12.7 mM hippuric acid which represented 3412 mg hippuric acid/1.5 liters. A total daily dose of 3000 mg Aqua Bimini™ as quinic acid ammonium chelate/70 kg person is equivalent to 43 mg/kg or equal to about 43 mg/liter=64.5 mg/1.5 liters. The total water volume of the human body was estimated to be 0.7 liters/kg (provided Aqua Bimini™ is equally distributed in water and urine); then 0.7 liters×70 kg liters=49 liters in which 3000 mg is dissolved is equivalent to 3000 mg/49 liters=91.8 mg/1.5 liters that should have been present if quinic acid was completely absorbed and not excreted.

Thus these calculations support that 64.5 mg/1.5 liter divided by 91.8 mg/1.5 liter or about only 70.3% of the daily administered dose of Aqua Bimini™ (i.e. quinic acid at 3000 mg/ml) was excreted into urine as hippuric acid on a daily basis, and the remaining 29.7% of Aqua Bimini™ was either absorbed into systemic circulation as hippuric acid or further metabolized to other compounds.

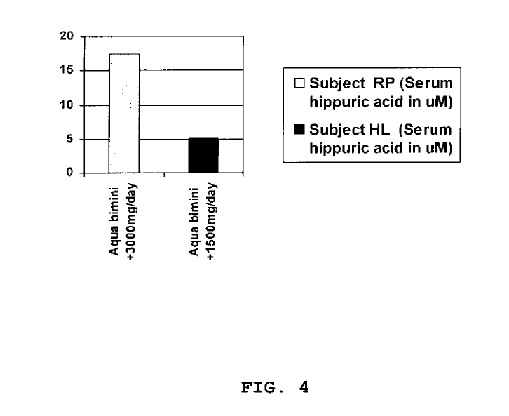

Yet only mM amounts of hippuric were found in urine-only extremely low levels of hippuric acid <20 μM were found in serum (see FIG. 4). Hippuric acid was barely detectable in serum within this dose range, thus, one of ordinary skill in the art can appreciate that such results correlate with the poor absorption of quinic acid into systemic distribution and its conversion therein to hippuric acid. In addition, at least the difference in serum concentration (i.e. <0.02 mM, FIG. 4) and urine levels of hippuric acid after Aqua Bimini™ administration of 3000 mg/day, (0.49-12.7 mM, FIG. 2, Table 3, and text calculations) establish that at least some quinic acid was metabolized to other compounds and not only excreted as hippuric acid.

Even at these low levels of serum hippuric acid there was a dose response to Aqua Bimini™ treatment observed for this material (FIG. 4). Taken together these data of serum and urinary quinic/hippuric acid levels indicate very little quinic acid was found in systemic circulation (<0.1% of the administered dose, see FIG. 1 and text calculations), and although hippuric acid could be detected if both serum and urine proving that quinic acid was indeed subject to systemic uptake metabolism, about 70.3% of the administered dose was excreted unchanged in the urine (see FIG. 2 and text calculations).

Of the remaining 29.7% of Aqua Bimini™ unaccounted for in the urine, only trace amounts was identified as hippuric acid in serum, thus paralleling the pharmacokinetic data of quinic acid and hippuric in serum (see also Table 3 and FIGS. 1-4). Because only trace amounts of either quinic acid or hippuric acid were found in serum, then potentially only 29.7% of the daily administered dose of Aqua Bimini™ could be converted to other compounds in systemic circulation or the GI tract.

This pharmacokinetic analysis suggests that the previously documented efficacious effects of quinic acid were due to a precursor compound active in the shikimate pathway, rather than due to quinic acid itself.

### Example 3

### Dependence of Hippuric Acid Levels in Urine on the Corresponding Levels of Quinic Acid in Urine

In this study baseline values for urinary values for quinic acid, hippuric acid, tryptophan and nicotinamide were determined in a broad-based reference population as well (n=9) between 12-86 years of age. The data for quinic and hippuric acids are summarized in Table 3 with the individual quinic acid-treated urinary values presented in FIGS. 2-3). There was a striking highly significant linear correlation (p<0.001) between the urinary levels of quinic and hippuric acids based on the entire sampled population of controls (unexposed) and repeat sampled Aqua Bimini™ (quinic acid) treated individuals (n=45). This was taken as strong evidence that hippuric acid was mainly influenced by dietary factors associated with quinic acid in colored fruits and vegetables and did not reflect environmental toxic aromatic exposures such as benzene or toluene.

### Example 4

### Serum Protein Thiol Analyses as In Vivo Estimates of Individual Antioxidant Status

After the pharmacokinetic data of quinic acid and hippuric acid reported on here, it was apparent that previously used oral doses of quinic acid were not responsible for any efficacy mediated directly by quinic acid itself; i.e. because it could not be detectable in systemic circulation, and about 40-60% of the quinic acid dose was excreted unmetabolized in the urine albeit over a 9 month (Table 4 and text calculations). Although the early reports were evaluated against varied clinical endpoints including (i) recovery from doxorubicin chemotherapeutic treatment and (ii) growth arrest without cell death and cell survival, we have noted that both these events were very dependent on redox (oxidation/reduction) balance and DNA repair. Consequently, here we have used the status of serum protein thiols as the indicator of antioxidant efficacious effects.

Metabolic fate of Aqua Bimini™ (quinic acid ammonium chelate) over a 9 month period in man involving 1 month treatment and 8 months of follow-up (no treatment) are provided in Table 4.

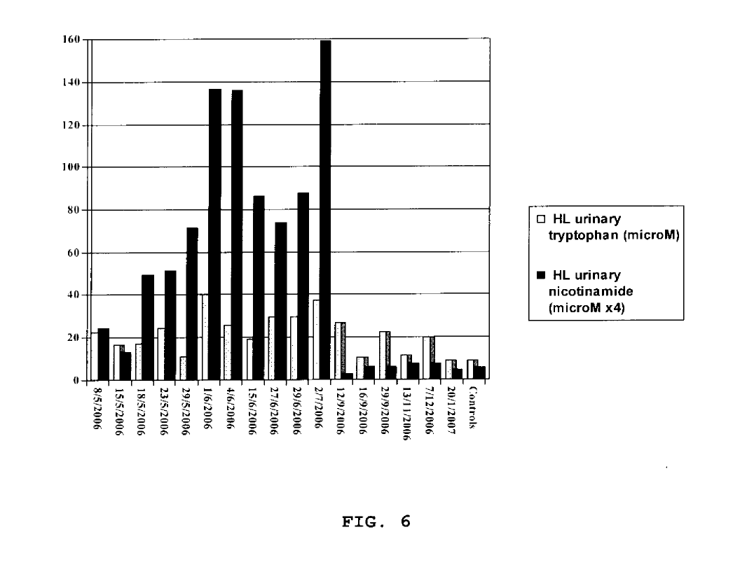

The serum protein thiol data are summarily presented in FIG. 6-8. First, FIGS. 6-7 present the individual data points distinguishing the before (baseline) serum thiol values from the after intervention values including both the short term dryout period from June 2 to August 31, and also a longer term dryout period (September 2006 to Jan. 20, 2007).

TABLE 4

| | | | Dose | |
|---|---|---|---|---|
| | Total dose | Total dose | recovery | Dose storage/ |
| Oral dose | for 9 months | excreted in | (excrete/ | metabolism |
| quinic acid | administered | 9 months^{a} | dose) | (100%-recovery |
| Subject RP | | | | |
| 3000 mg/day | 90 gm | 53 gm | 59% | 41% |
| Subject HL | | | | |
| 1500 mg/day | 45 gm | 18.5 gm | 40% | 60% |
| Ratio | 2.0 | 2.86 | — | — |

^{a}The average quinic acid urinary levels were calculated from the total data displayed in FIG. 3-4 and summarized in Table 3 as the mean for the entire sampling period of 9 months as: RP = 184 mM, n = 19 or 53 gm/1.5 liters; HL = 64.4 mM, n = 17 or 18.5 gm/1.5 liters.

It was apparent that Aqua Bimini™ supplementation was associated with a significant increase in reduced thiols (—SH) being present in the proteins, that even remained elevated throughout the treatment and follow-up periods. This was considered direct evidence that the redox balance was shifted away from the oxidant state. Because serum proteins contain signal transducing proteins, this was strong evidence of a major shift toward the antioxidant state occurring in vivo during supplementation with Aqua Bimini™, and remarkably for at least a 9 month evaluation period.

It was concluded that although Aqua Bimini™ was a powerful antioxidant, it must be causing these effects as a prometabolite by being converted into other compounds that could mediate antioxidant effects. The logic was simply that since there was no systemic accumulation of quinic acid in serum even after repeat dosing of Aqua Bimini™ for 36 days and an additional 8 month follow-up period with no treatment, then how could quinic acid in itself raise serum thiols when there had been no further supplementation for 8 months (See FIGS. 6-7).

FIG. 8 further clarifies that an individual's baseline serum thiols varied from one individual to another, presumably because of genetic and environmental factors. However, when Aqua Bimini™ (quinic acid ammonium chelate) was orally administered, there was an intra-individual increase in serum thiol levels independent of the background levels (i.e. baseline thiol status).

As a result, it was concluded that the antioxidant efficacious effects of Aqua Bimini™ were clearly present, and yet not caused by quinic acid itself, but rather by other metabolism it induced.

### Example 5

### Urinary Analysis of Metabolites Associated with the Microbial/Plant Shikimate Pathway

Intestinal micro flora capable of metabolizing quinic acid at least to hippuric acid is well established since quinic acid when administered orally is identified as hippuric acid in urine samples, but when quinic acid was administered by intraperitoneal injection there was no conversion to hippuric acid.

Despite the hippuric acid evidence of a functional shikimate pathway existing in the human GI tract, there have been no studies reported demonstrating that quinic acid could lead to an increased production of the compounds being generated along the metabolic route from shikimate to chorismate to tryptophan to nicotinamide. For the first time we have determined the urinary levels of tryptophan and nicotinamide were that were induced by exposure to aromatic amino acid precursors such as Aqua Bimini™ (quinic acid ammonium chelate).

The GI tract is one of the most important organ systems of the body, responsible for breakdown and re-synthesis of proteins, fats and sugars necessary to maintain proper nutrition for cellular growth and health maintenance in the rest of the body. Some of the main nutritive sources not synthesized by the body, but much needed for signal transduction (e.g. via serotonin and dopamine) and building proteins are the aromatic amino acids tryptophan, phenylalanine and tyrosine. They are all produced by the shikimate pathway along with nicotinamide and NAD, a primary energy source.

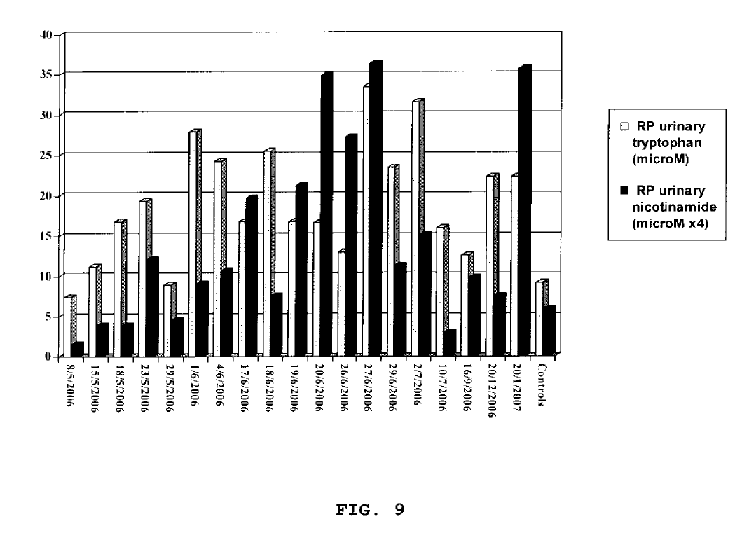

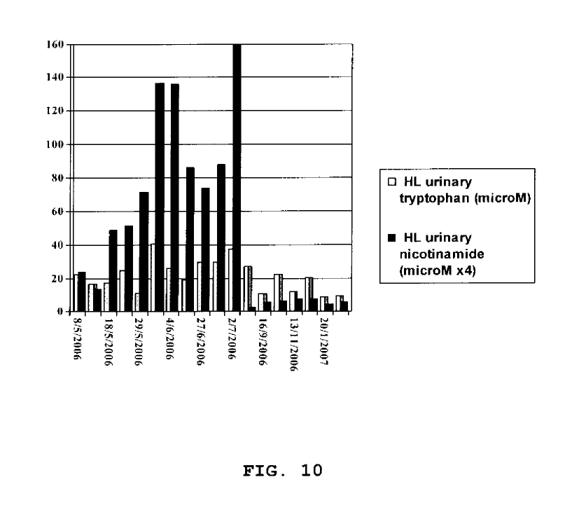

The data analysis presented in Table 3 and FIG. 9-10 show that when urinary levels of tryptophan and nicotinamide were measured in two subjects, one receiving 1500 mg/day and the other 3000 mg/day for 33 consecutive days (i.e. treatment period), both doses had significantly elevated levels of urinary tryptophan and nicotinamide, when compared to controls (unexposed). Even when samples were included in the analysis after 8 months of follow-up, the tryptophan and nicotinamide levels in urine still remained elevated. Tryptophan and nicotinamide in urine are well-known strong indicators of the status of these nutrients in the rest of the body, and so it was concluded that quinic acid supplementation not only increased the synthesis of hippuric acid in the GI tract but also tryptophan and nicotinamide.

In addition, both tryptophan and nicotinamide remained elevated in urine throughout the follow-up period of 8 months, which was support for their involvement in mediating the antioxidative systemic effects as evidenced by increased serum thiols during the same period (See FIGS. 6-8).

### Example 7

### Analysis of Tryptophan, Nicotinamide in Relation to Serum Thiol

In an effort to better substantiate the relationship between tryptophan/nicotinamide and systemic antioxidant levels assessed by serum thiols, we have further considered analyzing the molar ratios of tryptophan or nicotinamide in urine in relation to the micromolar levels of serum thiols. Table 5 clearly shows the ratio of either tryptophan or nicotinamide increased levels in urine (excretion) compared to the increased micromolar levels of thiols in serum (uptake into systemic circulation), there was a strong dose response to quinic acid oral supplementation.

Remarkably, these ratios estimated the metabolic control of individual antioxidant status linked to Aqua Bimini™ (quinic acid ammonium chelate) consumption in accordance with the theoretical difference in dose supplementation was 3000 mg per day/1500 mg per day=2.0 ratio; experimentally calculated from Table 5 as tryptophan was 1.94/0.81=2.4 ratio; experimentally calculated from Table 3 as nicotinamide was 4.29/2.32=1.85 ratio.

The relationship of nicotinamide and tryptophan to serum protein thiol metabolism are provided in Table 5. The data are calculated from FIG. 6-8 and Table 3. A beneficial increase of at least 30% is preferred.

TABLE 5

| | | | |
|---|---|---|---|
| Oral dose | Increase in urinary | | Increase nicotinamide. |
| administered | quinic acid (QA) | Increase in serum | or tryptophan/ |
| for 36 days | metabolite (ratio)^{a} | protein thiols (ratio)^{b} | increase thiols^{c} |

Urinary Nicotinamide analysis (μM/μM)

| | | | |
|---|---|---|---|
| HL 1500 mg/day QA | 215/76 = 2.83 | 1.22 | 2.32 |
| (Aqua Bimini ™) | | | |
| RP 3000 mg/day QA | 57.9/10.7 = 5.41 | 1.26 | 4.29 |
| (Aqua Bimini ™) | | | |

Urinary Tryptophan analysis (μM/μM)

| | | | |
|---|---|---|---|
| HL 1500 mg/day QA | 21.3/19.3 = 0.99 | 1.22 | 0.82 |
| (Aqua Bimini ™) | | | |
| HL 3000 mg/day QA | 22.7/9.32 = 2.44 | 1.26 | 1.94 |
| (Aqua Bimini ™) | | | |

^{a}Ratio average nicotinamide or tryptophan urinary levels in uM during treatment + follow-up divided by the levels at start of treatment: dates 8 May 2006 and 15 May 2006. Total data are presented in FIG. 9-10 and Table 3).
^{b}Calculated as the ratio in the after to before (baseline) Aqua Bimini ™ treatment values presented in FIG. 8.
^{c}Baseline Ratio (non-supplemented values) of ^{a} (increase in tryptophan or nicotinamide) to ^{b} (increase thiols) = ^{c} which is to about 1.0/1.22 = 0.82.

### Example 8

### Indolediamine Oxygenase Activity as Measured by Kynurenine

Once tryptophan synthesis has been stimulated by the shikimate pathway, it is then further modified by catabolic metabolism via indolediamine oxygenase (IDO) first to kynurenine and/or hydroxtryptophan-serotonin and/or to quinoline-dopamine or to nicotinamde-NAD (energy). The inhibition of IDO activity by quinic acid exposure in vivo favors reduction in immunosuppressive activity.

IDO activity was calculated by determining the molar ratios of tryptophan to kynurenine in 200 ul aliquots of 2 M TCA-precipitated serum by high pressure liquid chromatograph (HPLC). The HPLC method has already been published in all its details except the instrument was a HP 1100 series and the column was a XTERRA MSC18 (3.5 cm long, 4.6 mm×50 mm) (Laich, A, Neurauter, G, Widner, B, Fuchs, D. More rapid method for simultaneous measurement of tryptophan and kyurenine by HPLC. Clinical Chemistry 48 (3): 579-580, 2002).

The effect of quinic acid was further characterized on the end products of the shikimate pathway. Accordingly, quinic acid supplementation was analyzed against IDO activity through simultaneous detection of tryptophan and kynurenine. A high ratio of tryptophan/kynurenine is immunosuppressive (Bauer, T M, Jiga, L P, Chuang, J-J, Randazzo, M, Opelz, G, Terness, P Studying the immuno-suppressive role of indoleamine 2,3-dioxygenase: tryptophan metabolites suppress rat allogeneic T-cell responses in vitro and in vivo. Transplant International 18 (2005) 95-100, 2004). Quinic acid inhibits IDO activity because tryptophan levels are increased rather than being decreased by metabolism to kynurenine. These data suggests that quinic acid itself is not the efficacious form of quinic acid activity.

### Example 9

### Animal Studies

C57BL/6 female mice were fed 4 mg/ml C-Med-100® or 2 mg/ml quinic acid dissolved in autoclaved tap water for 21 days. The animals were sacrificed, spleens removed and blood collected. The cell samples were analyzed in a Sysmex KX-21N (Sysmx Corporation, Kobe, Japan) and plasma/serum samples prepared for chemical analysis by HPLC for quinic acid and tryptophan.

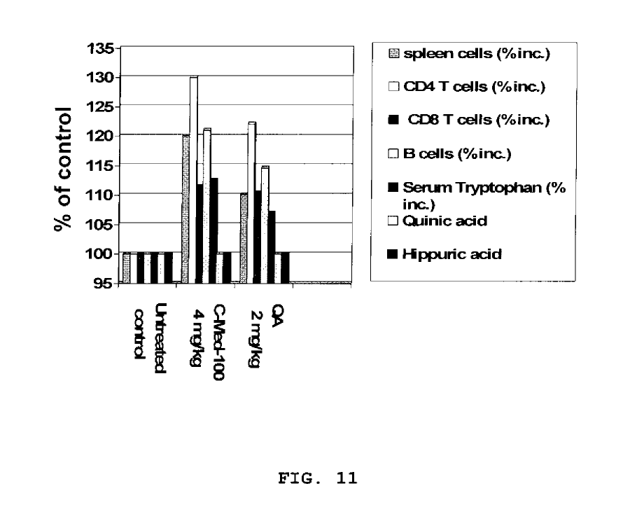

As seen from Table 6 differential lymphocyte counts in peripheral blood samples of mice after 21 days supplementation with precursor to an aromatic acid increased about as much as did the mouse serum tryptophan levels. FIG. 11 shows an in vivo estimation of blood cells and serum tryptophan after 21 days treatment and levels of hippuric acid (<0.02 ug/ml) and quinic acid (<2 mg/ml) were also simultaneously determined, but found to be below HPLC detection when quinic acid was dosed at 1 mg/ml in drinking water of mice or about 500 mg/kg. It is clear from Table 6 and FIG. 11 that the administration of quinic acid leads to increased serum tryptophan levels determined in vivo in the mouse, but no quinic acid was detectable, and yet s use still caused an immunostimulatory response.

As shown herein throughout the entire specification quinic acid chelates enhances aromatic amino acid production in vivo. Table 7 below provides a direct pharmacokinetic comparison of orally administered Aqua Bimini™ to doses of tryptophan already known to have therapeutic value in animals including humans. Such comparison provides an unexpected increase of aromatic amino acid levels when a precursor of aromatic amino acid are used orally.

TABLE 6

| Treatment | | | In vivo | | | In vivo plasma tryptophan | | |
|---|---|---|---|---|---|---|---|---|
| after day 21 | Estimated | | spleen cells ×10⁶ | | | | mean ± s.d. | |
| intervention | daily dose | | n | mean ± s.d. | t-test | n | (μM) | p value |
| Controls | 0 | | 21 | 90 ± 10^{a} | — | 6 | 74.5 ± 5.7^{a} | — |
| (sterile water) | | | | | | | | |
| C-Med- 100 ® | 500 | mg/kg | 24 | 108 ± 11^{b} | p < 0.002 | 6 | 84.4 8.0^{b} | P < 0.036 |
| (4 mg/ml) | | | | | a vs b | | | a vs b |
| Quinic acid | 250 | mg/kg | 22 | 100 ± 10^{c} | p < 0.018 | 4 | 79.4 ± 2.7^{c} | p < 0.11 |
| (2 mg/ml) | | | | | a vs c | | | a vs c |

TABLE 7

| | | Urinary excretion | | |
|---|---|---|---|---|
| Nutritional | Oral Human | (Systemic metabolic indicator) | Clinical | |
| supplement | dose used | daily sample (avg mg/liter) | indication | Reference |
| Aqua Bimini ™ | 21-42 mg/kg | Tryptophan. = 4.2 mg/liter^{b} | Antioxidant inc. | This study |
| (QA NH₄+)^{a} | (1500-3000 mg/day) | | DNA repair | |
| (33 days) | | | | |
| Therapeutic | 100 mg/kg | Tryptophan. = 1.3 mg/liter^{c} | Obesity | Cavaliere⁴⁰ |
| tryptophan | (50-200 mg/kg) | | Anorexia | Cavaliere⁴⁰ |
| | | | Migraine | Gedye⁴¹ |
| | | | Depression | Chouinard⁴² |
| | | | Insomnia Monkeys | Brown⁴³ |
| | | | (50-200 mg/kg) | |
| | | | self-biting mice | Weld⁴⁴ |
| | | | (125 mg/kg) | |
| | | | Depression | Wong⁴⁵ |
| | | | Pigs stress | Li⁴⁶ |

^{a}QA NH₄+ = quinic acid ammonium chelate (Quinmax), trypto. = tryptophan, avg = average, inc. = increased
^{b}Calculated from the average urinary levels of trypophan reported for a 9 month evaluation period in Table 3. Subject RP (3000 mg/day) = 19 ± 7 μM and Subject HL (1500 mg/day) = 22 ± 9 μM; Avg = 20.5 μM or 4.2 mg/liter urine
^{c}Calculation from literature: Smith, D F. Effects of age on serum tryptophan and urine indicant in adults given a tryptophan load test. Eur Drug Metab Pharmakinet 1982; 7 (1): 55-58.

### REFERENCES

^{d}Cavaliere, H, Medeiros-Neto, G. The anorectic effect of increasing doses of L-tryptophan in obese patients. Eat Weight Disord 1997; 2(2): 211-215
^{e}Gedye, A. Hypothesized treatment for migraines using low doses of tryptophan, niacin, calcium, caffeine, and acetylsalicyclic acid. Med Hypothesis 2001; 56(1): 91-94.
^{f} Chouinard, G, Young, S N, Annabie, Sourkes, T L. Tryptophan-nicotinamide, imipramine and their combination in depression. A controlled study. Acta Psychiatr Scand 1979; 59(4): 395-414.
^{g}Brown, C C, Horrom, N J, Wagman, A M. Effects of L-tryptophan on sleep onset insomniacs. Waking Sleeping 1979; 3(2): 101-108.
^{h}Weld, K P, Mench, J A, Woodward, R A, Bolesta, M S, Suomi, S J, Higley, J D. Effect of tryptophan treatment on self-biting and central nervous system serotonin metabolism in rhesus monkeys (Macca mulatta). Neuropsychophamacology 1998; 19(4): 314-321.
ⁱWong, P T, Ong, Y P. Acute antidepressant-like antianxiety-like effects of tryptophan in mice. Pharmacology 2001; 62(3): 151-156.
^{j}YK, Kerr, B J, Kidd, M T, Gonyou, H W. Use of supplementary tryptophan to modify behavior of pigs. J Animal Sci 2006; 84 (1): 212-220.

Every patent and non-patent publication cited in the instant disclosure is incorporated into the disclosure by reference to the same effect as if every publication is individually incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

1. A precursor for an aromatic amino acid having the following formula:
A-(X)ₙ
where A is selected from the group consisting of 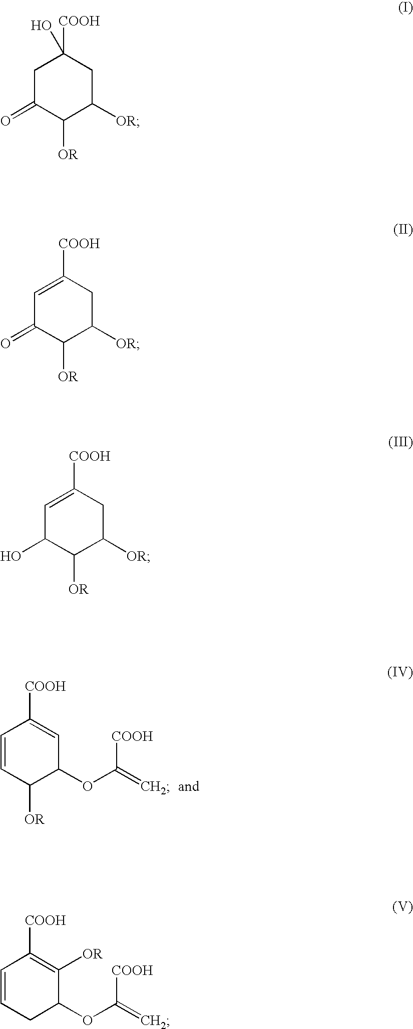
and anions, esters thereof; wherein R is H, C₁-C₃ alkyl, and
X is selected from the group consisting of any monovalent, divalent, trivalent anions that can form salts or hydroxides or amines, and
“n” is an integer or a fraction, wherein 1<n=10.

2. A composition comprising (a) a therapeutically effective amounts of a compound selected from the group consisting of 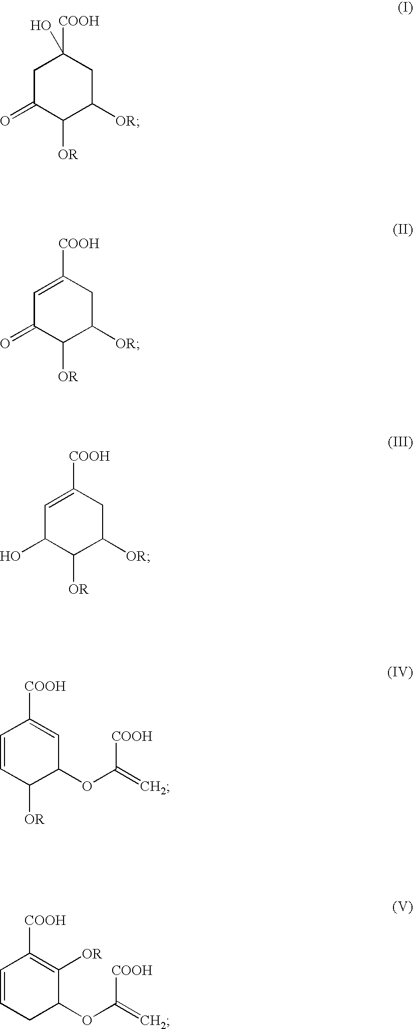
wherein R is H, or C₁-C₃ alkyl; and anions and esters thereof, and
A-(X)ₙ (VI)
wherein A, X and n are as defined in claims 1; and
(b) a pharmaceutically acceptable vehicle.

3. The composition of claim 2, wherein the composition further comprise (c) therapeutically effective amounts of a primary anti-oxidant selected from the group consisting of nicotinamide, niacin, flavonoids, cinnamic acid or derivatives thereof, ascorbic acid or derivatives thereof, tocopheral or derivatives thereof, and vitamin A or D or derivatives thereof; and (d) an optional ingredient selected from the group consisting of anti-neoplastic agents, anti-infectives, anti-depressants, mood-enhancing agents, anti-inflammatory agents, immune modulators, and a secondary antioxidant.

4. The composition of claim 2, wherein the therapeutically effective amount of the precursor for an aromatic amino acid is in the range of about 250 mg/day to about 5000 mg/day.

5. The composition of claim 2, wherein the aromatic amino acid is selected from the group consisting of tryptophan, phenylalanine, tyrosine and phenylephederine.

6. The composition of claim 3, wherein said primary anti-oxidant is a flavonoid selected from the group consisting of quecrcitin, rutin, chrysin, myricetin, genisten, hesperidine, naringin, and mixtures thereof.

7. The composition of claim 6, wherein said optional ingredient is a secondary antioxidant and is selected from the group consisting of coenzyme Q, pyruvate, coenzyme A, ubiquinol, NADH, NAD, NADP, NADPH, adenine, adenosine, niacin, nicotinamide, campesterol, catechin, chlorogenic acid, cinchonain, corynantheine, corynoxeine, daucosterol, epicatechin, harman, hirsuteine, hirsutine, iso-pteropodine, loganic acid, lyaloside, mitraphylline, oleanolic acid, palmitoleic acid, procyanidins, pteropodine, quinovic acid glycosides, rhynchophylline, sitosterols, speciophylline, stigmasterol, strictosidines, uncarines, and vaccenic acid.

8. The composition of claim 3, wherein said optional ingredient is present and is selected from the group consisting of anti-neoplastic agents, anti-infectives, anti-depressants, mood-enhancing agents, and anti-inflammatory agents.

9. The composition of claim 8, wherein said anti-neoplastic agents are selected from the group consisting of fluorinated pyrimidines, cytidine analogues, purine antimetabolites, plant alkaloids, alkylating agents, antracene derivatives, antitumor antibiotics, metal complexes, anti-aminophospholipid antibodies, anti-angiogenic agents, and radiotherapeutics.

10. The composition of claim 3, wherein said optional ingredient is an anti-infective selected from the group consisting of sulfonamides, penicillins, cephalosporins, aminoglycosides, protein synthesis inhibitors, antifungals, antiviral agents, anti-tuberclosis agents.

11. The composition of claim 3, wherein said optional ingredient is an anti depressant selected from the group consisting of tricyclic anti-depressants, and serotonin reuptake inhibitors.

12. The composition of claim 3, wherein said optional ingredient is an anti-inflammatory agent selected from the group consisting of steroidal anti-inflammatory agents, and non-steroidal anti-inflammatory agents.

13. The composition of claim 2, wherein said composition is in the form of a capsule, a tablet, a syrup, a liquid, an emulsion, an elixir for oral administration.

14. An animal model methodology for identifying an effective anti-oxidant therapeutic regimen for a disease state characterized by aromatic amino acid deficiency comprising the steps:
(a) obtaining a mammal selected from the group consisting of mice, rats, rabbits, dogs, pigs, monkeys and humans,
(b) determining a baseline value ratio for urinary tryptophan, nicotinamide, hippuric, kynurenine levels/tissue or blood thiol levels of said mammal,
(c) administering to the mammal a composition comprising a precursor of an aromatic amino acid,
(d) determining a value for the ratio of urinary tryptophan, nicotinamide, hippuric, kynurenine levels/tissue or blood thiol levels of said mammal after a cycle of the anti-oxidant treatment,
(e) comparing the values obtained in steps (b) and (d), wherein a 30% increase over the baseline value indicates that the therapeutic regimen has been effective.

15. The method of claim 14, wherein the tissue thiol(s) sample is selected from the group consisting of plasma protein, blood protein, and serum protein, or a 30% to 80% saturated ammonium sulfate precipitated fraction of plasma protein, blood protein, and serum protein.

16. The method of claim 14, wherein the animal exhibits symptoms of a disease similar to those in humans.

17. The method of claim 14, wherein the value obtained in steps (b) and (d) are for the ratio of urinary tryptophan, nicotinamide, kynurenine/serum protein thiols of said mammal.

18. A method of testing the in vivo oxidative stress in a patient in need comprising
(a) treating the patient with a composition comprising a precursor for an aromatic amino acid,
(b) obtaining a sample of protein thiols from said patient,
(c) determining the protein thiols levels in the sample,
(d) measuring urinary levels of tryptophan or nicotinamide,
(e) analyzing the thiol(s) levels in sample of step (c) in relation to the urinary levels of tryptophan or nicotinamide obtained in step (d) expressed as (d)/(c),
wherein an increase in the value of said ratio is a predictor of low oxidative stress and down regulation of DNA repair.

19. The method of claim 18, wherein the sample of protein thiol(s) is selected from a group consisting of plasma, blood, and serum samples.

20. A method of ascertaining a patient's susceptibility to a disease characterized by increased level of oxidative stress comprising
(a) treating the patient with a composition comprising an aromatic amino acid precursor,
(b) obtaining a body sample from said patient comprising protein thiol(s) levels,
(c) determining the protein thiol(s) levels of the sample,
(d) measuring urinary levels of tryptophan or nicotinamide levels,
(e) analyzing the thiol(s) levels in sample of step (c) against the urinary levels of tryptophan or nicotinamide obtained in step (d) wherein a higher value against a predetermined normalized value obtained from a population suffering from said disease is the predictor of said individual to oxidative stress and down regulation of DNA repair.

21. The method of claim 20, wherein the body sample comprising protein thiol(s) is selected from a group consisting of plasma sample, blood sample, serum sample.

22. A method for prophylactically treating a condition characterized by deficiency in serum aromatic amino acid levels comprising administering the composition of claim 2.

23. The method of claim 22, wherein the condition is characterized by exhibiting the symptoms generalized weakness, low energy, inability to focus and inability to be alert.

24. The method of claim 22, wherein the condition is selected from the group consisting of depression, mood disorders, Alzheimer, dementia, Parkinson disease, memory loss, ADHD.

25. The method of claim 22, wherein the condition is selected from the group consisting of leucopenia, hypothyroidism, bloodshot eyes, cataracts.

26. The method of claim 22, wherein the condition is selected from the group consisting of crohns disease, inflammatory bowl disease, rheumatoid arthritis, osteoarthritis, generalized eczema, psoriasis, vitiligo and decreased fertility.

27. The method of claim 22, wherein said composition is combined with food.

28. The method of claim 22, wherein said composition is a food containing therapeutic amounts of an amino acid precursor resulting in an increase in antioxidant activity of >25% when measured by serum thiol(s) status.

29. A method for determining the risk for developing a disease characterized by deficiency in aromatic amino acids in an individual in need thereof, comprising the steps of:
(a) obtaining levels of the individual's serum thiol(s) and levels of urinary tryptophan, nicotinamide or combinations or both,
(b) establishing a value for the ratio of serum thiol(s)/urinary tryptophan, nicotinamide or combinations for said individual,
(c) comparing the value of step (b) against a predetermined normalized levels of the same obtained from a population afflicted by said disease, wherein said comparison indicates the baseline value for the oxidative stress in said individual.

30. The method of claim 29, wherein the serum thiol is serum protein thiol.

31. The method of claim 29, further comprising the step (d) administering to the individual a composition comprising a precursor for an aromatic amino acid during a treatment cycle.

32. The method of claim 29, further comprising the steps (e) obtaining levels of the individual's serum protein thiol(s) and levels of urinary tryptophan, nicotinamide, hippuric acid, or all, after the end of the treatment cycle, and (f) establishing a value for the ratio of urinary tryptophan, nicotinamide or combination thereof/serum protein thiol(s) for the individual, after said treatment cycle.

33. The method of claim 30, further comprising the step (g) comparing the value of step (f) with the baseline and the predetermined normalized levels of a population afflicted from said disease to establish the efficacy of the antioxidant therapy.

34. The method of claim 33, wherein the efficacy of the antioxidant therapy is determined by at least a 25% increase in the value obtained in step (c).

35. The method of claim 33, wherein the efficacy of the antioxidant therapy is determined by at least a 30% increase in the value obtained in step (c).

36. The method of claim 33, wherein steps (a)-(g) may be repeated for additional treatment cycles.

37. The method of claim 31, wherein the composition further comprise a secondary anti-oxidant compound and an optional ingredient.

38. The method of claim 37, wherein the secondary anti-oxidant compound is selected from the group consisting of nicotinamide, niacin, NAD, NADP, flavonoids, cinnamic acid or derivatives thereof, ascorbic acid or derivatives thereof, tocopheral or derivatives thereof, and vitamin A or D or derivatives thereof; a pharmaceutically acceptable vehicle.

39. The method of claim 31, wherein the composition further comprise an optional ingredient selected from the group consisting of anti-neoplastic agents, anti-infectives, anti-depressants, mood-enhancing agents, anti-inflammatory agents, immune modulators, and a secondary antioxidant, or mixtures thereof.

40. The method of claim 29, wherein the disease is selected from the group consisting of depression, generalized weakness, generalized pain, autoimmune disorders, cancer, diabetes, and advanced age.

41. The method of claim 29, wherein the treatment cycle ranges between disease is selected from the group consisting of depression, generalized weakness, generalized pain, autoimmune disorders, cancer, diabetes, and advanced age.
